(19) United States
(12) Patent Application Publication
(10) Pub. No.: US 2010/0104643 A1
(43) Pub. Date: Apr. 29, 2010

(54) PHARMACEUTICAL COMPOSITIONS

(75) Inventors: Ian Wilding, Nottingham (GB); Russell Pendleton, London (GB)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignee: ZYSIS LIMITED, Beeston, Nottingham (GB)

(21) Appl. No.: 12/595,770

(22) PCT Filed: Apr. 14, 2008
(86) PCT No.: PCT/GB2008/001306

§ 371 Date: Dec. 18, 2009

(30) Foreign Application Priority Data
Apr. 13, 2007 (GB) 0707127.7

(51) Int. Cl. A61K 9/14; A61K 31/55; A61P 25/24

(52) U.S. Cl. 424/488; 514/217; 424/484

(57) ABSTRACT

The present invention provides an orally deliverable pharmaceutical composition for the once-daily (OD) administration of trimipramine. The composition comprises a therapeutically effective amount of trimipramine and at least one pharmaceutically acceptable excipient. The compositions of the invention may exhibit one or more of the release profiles defined in this specification.

The invention relates to pharmaceutical formulations comprising trimipramine. More particularly, the invention relates to orally deliverable pharmaceutical compositions for the controlled release of trim ipramine.

Trim ipramine is 10,11-Dihydro-5-(3-dimethylamino-2-methylpropyl)-5H-dibenz(b,f) azepine and has the following structure:

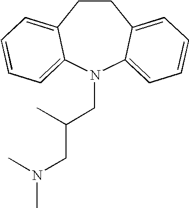

Trimipramine is used to relieve symptoms of depression such as feelings of sadness, worthlessness, guilt, loss of interest in daily activities, changes in appetite, tiredness, sleeping too much, insomnia, and thoughts of death or suicide. Trimipramine is a tricyclic antidepressant (TCA) and for many years it was thought that tricylic antidepressants work by inhibiting the re-uptake of the neurotransmitters norepinephrine and serotonin (5-HT) by nerve cells. However, this response occurs immediately, yet mood does not lift for around two weeks. It is now thought that changes occur in receptor sensitivity in the cerebral cortex and hippocampus. The hippocampus is part of the limbic system, a part of the brain involved in emotions. Presynaptic receptors are affected: a1 and b1 receptors are sensitized, a2 receptors are desensitised (leading to increased noradrenaline production). As a summary, trimipramine acts by decreasing the reuptake of norepinephrine and serotonin (5-HT).

The listing or discussion of a prior-published document in this specification should not necessarily be taken as an acknowledgement that the document is part of the state of the art or is common general knowledge.

Trimipramine is metabolized to the main metabolites desmethyltrimipramine, didesmethyltrimipramine, 2-hydroxy trimipramine and 2-hydroxy desmethyltrimipramine. Desmethyltrimipramine, the major primary demethylated metabolite, is considered to show pharmacological activity similar to the demethylated metabolites of other tricyclic antidepressants. The upper limit of the therapeutic trough plasma concentrations is thought to be 0.24 mg/l for trimipramine and 0.38 mg/l for desmethyltrimipramine but there are only scarce data on the concentration response relationship in trimipramine antidepressant treatment (Moffat A C, Jackson J V, Widdop B, Clarke's Isolation and Identification of Drugs, 2nd edition. London: Pharmaceutical Press; 1986).

Demethylation of trimipramine appears to be catalyzed at least partly by CYP2C19 since individuals lacking CYP2C19 activity had high concentrations of trimipramine but low concentrations of the demethylated metabolite. CYP2D6 polymorphisms were shown to cause an extensive variability in trimipramine pharmacokinetics with strong effects both, on first pass metabolism as well as on systemic elimination. The mean systemic clearances in ultrarapid metabolizers of CYP2D6 substrates were 2.5-fold higher than in poor metabolizers, and bioavailability differed even 6-fold between poor and ultra-fast metabolizers resulting in an about 15-fold difference in total oral clearance with extremes as low as 3.5 l/h in the poor metabolizer group and as high as 712.6 l/h in the ultrarapid metabolizer group (Kirchheiner et al, Trimipramine pharmacokinetics after intravenous and oral administration in carriers of CYP2D6 genotypes predicting poor, extensive and ultrahigh activity, Pharmacogenetics, 2003 December; 13(12):721-8).

To consider the effects of the CYP2D6 polymorphism in traditional immediate release (IR) trimipramine treatment, individual dosages can be modified according to the differences in clearances caused by the CYP2D6 genotype. Trimipramine has a linear dose-concentration relationship for doses up to 150 mg, whereas desmethyltrimipramine already shows a deviation from linearity within the therapeutic dose range. Lacking CYP2D6 activity might lead to early saturation of the remaining enzymatic pathways, and, thus, nonlinear increase of plasma concentrations even at smaller doses can be anticipated. Whereas pharmacokinetic differences caused by genotypes can be compensated by dose adaptations, the pharmacodynamic consequences expected in patients are more complex and pharmacokinetic variability explains only part of the clinically observed variability in response and adverse events.

The recommended initial dose is 75 mg daily in two or three divided doses. Initial tolerance may be tested by giving the patient 25 mg on the evening of the first day. The initial dose can be increased by 25 mg increments, usually up to 150 mg daily, preferably by adding to the late afternoon and/or bedtime doses. The greater part of the daily dose should be given in the late afternoon or at bedtime to minimize bothersome daytime sedation. For adults with severe disease, a higher initial dose of 100 mg daily in two or three divided doses may be indicated. The usual optimal dose is 150 mg to 200 mg daily, but some patients may require up to 300 mg (or even 400 mg) daily, depending on tolerance and response of each individual patient (http://en.wikipedia.org/wiki/Trimipramine).

In the case of elderly or debilitated patients, it is considered advisable to give a test dose of 12.5 to 25 mg and after 45 minutes examine the patient sitting and standing to check for orthostatic hypotension. Initial doses should usually be no more than 50 mg a day in divided doses, with weekly increments of no more than 25 mg a week, leading to a usual therapeutic dose range of 60 to 150 mg a day. Blood pressure and cardiac rhythm should be checked frequently, particularly in patients who have unstable cardiovascular function. Once a satisfactory response has been obtained in all patients, the dosage should be adjusted to the lowest level required to maintain remission and avoid relapse.

There are a number of disadvantages associated with the conventional dosage regimen for trim ipramine described above for treating depression. Multiple dosing each day leads to significant fluctuation in the peak to trough ratio which significantly increases the chance of clinically relevant adverse events. In addition, market research suggests that patients much prefer oral medications that can be taken as infrequently as possible but with a regular, easy to remember pattern and that are well tolerated. Therefore, any reduction in dosing frequency will bring material improvements in patient convenience and compliance. In addition optimization would be desirable to ensure the dosing regimen is appropriate for the treatment of a combination of insomnia and depression. It would therefore be preferable to move from the current multiple dosing regimen to a dose regimen in which greater quantities of trimipramine can be administered in one dose, preferably without significantly increasing adverse events.

In addition, as a consequence of the wide variation in plasma exposure linked to the different genotypes of CYP2D6, then poor metabolisers could be receiving significantly greater drug exposure than required for efficacy and more importantly be experiencing unacceptable tolerance issues eg orthostatic hypotension associated with high peak plasma concentrations. In these situations it would be normal to reduce the dose but for patients with ultra-rapid metabolism then these patients would have a significant risk of not having a therapeutically effective level of trim ipramine in their plasma. In order to reduce these difficulties in the administration of trimipramine, the inventors arrived at the present invention.

The subject invention seeks to address the above-mentioned deficiencies by the provision of orally deliverable pharmaceutical compositions for the once daily (OD) administration of trimipramine, the compositions comprising a therapeutically effective amount of trimipramine and at least one pharmaceutically acceptable excipient. Such compositions may be used for both the treatment of depression and insomnia plus a number of other medical indications as described later in this specification.

Unless otherwise indicated herein, the term “trimipramine” refers to 10,11-Dihydro-5-(3-dimethylamino-2-methylpropyl)-5H-dibenz(b,f) azepine its pharmaceutically acceptable salts, and mixtures thereof. “Pharmaceutically acceptable salts” includes derivatives of trimipramine, wherein trimipramine is modified by making non-toxic acid or base salts thereof, and further refers to pharmaceutically acceptable solvates (including hydrates) of such salts. Examples of pharmaceutically acceptable salts include, but are not limited to, mineral or organic acid salts of the amine functionality of trimipramine. The pharmaceutically acceptable salts include non-toxic salts and the quaternary ammonium salts of trimipramine formed, for example, from organic and inorganic acids. Such salts include those derived from inorganic acid such hydrochloric, hydrobromic, hydroiodic, sulphuric, phosphoric, nitric, metal salts such as sodium salt, potassium salt and cesium salt, alkaline earth metal salts such as calcium salt and magnesium salt and combinations of the foregoing. Pharmaceutically acceptable organic salts include salts prepared from organic acids such as acetic, trifluoroacetic propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethanedisulfonic, oxalic, isethionic, HO₂C—(CH₂)ₙ—CO₂H (where n=0-4) and salts prepared from amino acids such as arginate, asparginate and glutamate. Trimipramine maleate is a preferred salt. The term “pharmaceutically acceptable salts” also includes mixtures of any of the foregoing derivatives of trim ipramine.

By the term “orally deliverable”, we include the meaning suitable for oral, including peroral and intra-oral (e.g. sublingual or buccal) administration. Preferably, the compositions of the invention are designed for peroral administration to a patient, i.e. by swallowing (e.g. eating or drinking).

By the term “once daily (OD) administration”, we include the meaning that compositions of the invention release the trimipramine in a controlled and/or modified manner over about 24 hours. This may include the meaning that the compositions of the invention exhibit an in vitro release profile wherein at least 40% of the trimipramine is dissolved within about 8 hours of placement in a standard dissolution test. For example, from about 50 to about 100% (such as from about 60, 70, 80 or 90 to 100%) of the trimipramine may be dissolved within about 8 to about 24 hours (such as from about 9, 10, 11 or 12 to about 16, 18, 20 or 22 hours) of placement in a standard dissolution test. Preferably, substantially all of the trimipramine is dissolved within about 24 hours of placement in a standard dissolution test. The term “once daily (OD) administration” is also intended to exclude conventional immediate release (IR) of trimipramine. In other words, the once daily compositions of the invention provide controlled and/or modified release of the trimipramine relative to conventional immediate release formulations.

The term “once daily (OD) administration” also includes once nightly administration. Compositions of the invention which are suitable for once nightly administration typically are intended to release trimipramine in a controlled and/or modified manner daily, but overnight (i.e. during the resting and/or sleeping hours of a patient/sufferer).

Unless otherwise indicated, as used herein, the term “standard dissolution test”, means a test conducted according to the “Paddle Method” at 100 rpm in 900 ml of a dissolution medium of aqueous 0.05M phosphate buffer (physiological pH range between 1 and 7) at 37° C., as described in the United States Pharmacopoeia, or other

The phrase “conventional immediate release (IR) of trimipramine” includes the meaning that substantially all of the trimipramine (contained in a dosage form) is released immediately, for example within 30 minutes of administration. In other words, such IR dosage forms typically have substantially no component which acts to control and/or modify (e.g. delay/sustain) the release of trim ipramine. This definition is intended to include the compositions of trimipramine described in the introductory pages of this specification which are currently typically used for the treatment of depression and insomnia.

By the term “controlled and/or modified (release)”, we include the meaning that after administration, release of the trimipramine is controlled and/or modified so that a dosage regimen in which trimipramine can be administered once daily can be provided. This may include prolonging and/or sustaining the release of trimipramine so that the time between doses of trimipramine can be increased to once daily. Such release may also be accompanied by a higher single dose of trimipramine in the compositions of the invention compared to the currently used immediate release formulations.

Typically, the compositions of the invention delay or prolong the release of a trimipramine dose so that after administration, the adverse event profile is reduced, or at least not significantly increased, compared to the current dosage regimen.

The modified/controlled once daily release characteristics of the compositions of the invention may be defined in relation to their in vitro or in vivo release profile or related values such as Cₘₐₓ, Tₘₐₓ and AUC, as described in more detail below.

A preferred embodiment of the invention is sustained/prolonged release OD compositions. These compositions, which are generally referred to herein as the sustained release OD compositions of the invention, are described in more detail below.

The compositions of the invention suitable for OD sustained release drug delivery may typically exhibit an in vitro release profile wherein on average from about 10 to about 50%, such as from about 15 to about 45%, for example from about 15 to about 30% of the trimipramine is dissolved within 3 hours after placement in a standard dissolution test.

The compositions of the invention which may be suitable for OD sustained release drug delivery may typically exhibit an in vitro release profile wherein on average from about 25 to about 100%, such as from about 30 to about 100%, for example from about 40 to about 100% or about 50 to about 100% of the trimipramine is dissolved within 8 hours after placement in a standard dissolution test.

The compositions of the invention which may be suitable for OD sustained release drug delivery may typically exhibit an in vitro dissolution rate after placement in a standard dissolution test wherein:
from about 5 to about 40% (e.g. from 10 to 30%) of the trimipramine is released after 2 hours;
from about 15 to about 70% (e.g. from 20 to 50%) of the trimipramine is released after 4 hours; and
50% or more (e.g. 60% or more) of the trimipramine is released after 8 hours.

Preferably, the in vitro release rate is independent of pH between 1 and 7.

The compositions of the invention which may be suitable for OD sustained release administration may typically exhibit an in vivo trimipramine plasma absorption profile following single dose oral administration wherein the time for 50% of the trimipramine to be absorbed into the plasma is from about 2 to about 12 hours, such as from about 3 to about 10 hours, for example from about 4 to about 9 hours or from about 5 to about 7 hours (e.g. about 6 hours).

The OD sustained release compositions of the invention may also be defined in terms of the amount of trimipramine which is released from the compositions in vivo at specified periods of time following oral administration. Such compositions may typically exhibit an in vivo release profile wherein:
from about 5 to about 40% (e.g. from 10 to 30%) of the trimipramine is released within 2 hours following administration;
from about 15 to about 70% (e.g. from 20 to 50%) of the trimipramine is released within 4 hours following administration; and
50% or more (e.g. 60% or more) of the trimipramine is released within 8 hours following administration.

The release characteristics of the sustained release OD compositions of the invention may be defined in relation to the peak plasma concentration (Cₘₐₓ) value of trimipramine when administered to human or animal patients. For example, the compositions of the invention which may be suitable for OD administration typically exhibit a trimipramine Cₘₐₓ value following oral administration of from about 10 to about 99%, such as from about 20 to about 80%, for example from about 30 to 60% of the Cₘₐₓ value achieved using a conventional immediate release (IR) dosage form of trimipramine when administered orally at an identical dose.

The release characteristics of the sustained release OD compositions of the invention may be defined by the ratio of the peak plasma concentration (Cₘₐₓ) of trimipramine to the plasma concentration of trimipramine 24 hours following administration (C₂₄) when administered to human or animal patients and prior to the administration of any further doses. The compositions of the invention typically exhibit a Cₘₐₓ to C₂₄ ratio, preferably under steady state conditions, that is less than about 4:1, preferably less than about 3:1, more preferably less than about 2.5:1, most preferably from 1.5:1 to about 2:1 (e.g. about 1:1).

The sustained/prolonged release OD compositions defined above comprise only one component of trimipramine. However, for a combined treatment of insomnia and depression, for example, it would be advantageous to ensure a second peak in the plasma profile during the hours of sleep in order to maintain the desired clinical effect during the night. As a consequence, a controlled release formulation capable of delivering the trimipramine as two or more distinct components separated is a preferred embodiment of the invention.

Therefore, the sustained/prolonged release OD compositions of the invention may comprise one or more additional components, e.g. a pulsed release component and/or an immediate release component (each comprising trimipramine). Such “multi-component” compositions are described in more detail below. This “multi-component” embodiment of the invention is described in more detail below.

The “multi-component” compositions of the invention typically exhibit an in vitro release profile comprising:

a first component wherein from about 20 to about 80%, such as from about 30 to about 70%, for example from about 40 to about 60% (typically about 50%) of the trimipramine (as a percentage of the total amount of the trimipramine in the composition, i.e. before any trimipramine is released) is dissolved within from about 0.5 to about 12 hours, such as from about 1 to about 10 hours, for example from about 2 to about 8 hours (typically, about 3, 4, 5, 6 or 7 hours) after placement in a standard dissolution test; and

a second component wherein from about a further 20 to about 80%, such as from about a further 30 to about 70%, for example from about a further 40 to about 60% (typically about a further 50%) of the trimipramine (as a percentage of the total amount of the trimipramine in the composition) is dissolved within from about 4 to about 24 hours, such as from about 6 to about 20 hours, for example from about 8 to about 16 hours (typically from about 10 to about 14 hours) after placement in the standard dissolution test.

The first component of trimipramine may be released as an initial immediate release bolus. Alternatively, the first component of trimipramine may be released as a sustained release component. The second component, which may be a pulsed or sustained release component, typically constitutes the remaining dose of trimipramine, and is released from the same formulation within the time periods defined above. Preferably, at least one of the first and second components is a sustained release component.

In one preferred embodiment, from about 10 to about 50% (such as from about 20 to about 40%, e.g. about 30%) of the trimipramine in the composition is released as an immediate release bolus. The remaining 50 to about 90% (such as from about 60 to about 80%, e.g. about 70%) is in the form of a sustained release component in which the remainer of the trimipramine is released in the time periods defined above in relation to the second component.

For example, the “multi-component” compositions of the invention may exhibit an in vitro release profile wherein on average 50% of the trimipramine is released as an immediate release bolus and the remaining does is released from the same formulation approximately four hours later after placement in a standard dissolution test.

In an alternative preferred embodiment, the first component defined above is a sustained release component. The remaining trimipramine in the composition (the second component) is in the form of a pulsed release component wherein the trimipramine is released (e.g. as an IR bolus or a further sustained release component) after the time periods defined above in relation to the second component.

The “multi-component” compositions of the invention may also exhibit an in vitro release profile comprising further components (i.e. more than two components in total) in which more trimipramine is dissolved.

For example, the in vitro release profile of the compositions of the invention defined above may further comprise a third component wherein from about a further 20 to about 60%, such as from about a further 25 to about 50%, for example from about a further 30 to about 40% of the trimipramine is dissolved within from about 6 to about 24 hours, such as from about 8 to about 20 hours, for example from about 10 to about 16 hours after placement in the standard dissolution test.

Additionally, the release profile of the “multi-component” compositions of the invention may further comprise a fourth component wherein from about a further 10 to about 40%, such as from about a further 20 to about 30% of the trimipramine is dissolved within from about 8 to about 24 hours, such as from about 10 to about 20 hours, for example from about 12 to about 16 hours after placement in the standard dissolution test.

The “multi-component” compositions of the invention defined above may typically exhibit an in vivo plasma absorption profile following single dose oral administration wherein the time for 50% of the trimipramine (based on the total amount of trimipramine in the composition) to be absorbed into the plasma is from about 0.5 to about 12 hours, such as from about 1 to about 10 hours, for example from about 2 to about 8 hours (typically, about 3, 4, 5, 6 or 7 hours). The remaining drug dose typically is absorbed in one or more further components so that the time for the remaining 50% of the trimipramine to be absorbed into the plasma is from about 4 to about 24 hours, such as from about 6 to about 20 hours, for example from about 8 to about 16 hours (e typically from about 10 to about 14 hours). Of course, this time will depend at least in part on the number of further components of drug release used.

As used herein, the phrase “plasma absorption profile” is intended to refer to the plasma concentration of trimipramine over time following administration to a human or animal patient. As known to those skilled in the art, the plasma absorption profile may be measured by deconvolution of controlled release pharmacokinetics versus an immediate release reference.

In the multi-component compositions of the invention described above, any combination of components may be used (e.g. any combination of the release profiles described above) such that the trimipramine is released in a controlled and/or modified manner over about 24 hours as described above in the definition of “once daily (OD) administration”.

The compositions of the invention may exhibit one or more of the controlled release profiles defined above.

The compositions of the invention comprise a therapeutically effective amount of trimipramine and at least one pharmaceutically acceptable excipient. In order to achieve one or more of the controlled release profiles described above, the therapeutically effective amount of trimipramine may be formulated in numerous different ways, including, but not limited to diffusion-controlled formulations (such as wax matrices or pellets), dissolution-controlled formulations (such press-coated formulations), dissolution/diffusion-controlled formulations, easily administrable formulations (such as chewable, fast dissolving, sprinkle or taste-masked formulations), enteric-coated formulations, osmotic pump technology formulations, tamper-resistant formulations, erosion-controlled formulations, ion exchange resins and combinations of the foregoing. The above formulations will be described in more detail below.

The sustained release OD compositions of the present invention may be provided as a programmed drug delivery formulation that delivers the beneficial agent after a predictable delay, the delay being independent of gastric emptying time, in the form of a tablet comprising a core comprising trimipramine and optionally one or more additional active agents, one or more pharmaceutical excipients that swell upon exposure to aqueous medium, and optionally other pharmaceutically acceptable excipients, wherein the core is coated with a water insoluble coating. A passageway may be drilled in the coat and covered with a band or a plug of a polymer composition that is soluble or swellable in the gastrointestinal fluids and whose water solubility is pH-independent. Upon erosion or dissolution of the soluble polymer the passageway is exposed and the fluid from the surrounding environment enters the system, causing it to swell and exert a pressure on the coat. The coat then ruptures to release the contents of the core. Alternatively, the core may be coated with a polymer composition that is insoluble but permeable to water, and the passageway may be coated with a water-insoluble pH-independent or pH-dependent polymer, preferably a pH-independent polymer. The water entering the core through the permeable membrane causes the core to swell and the swelling exerts a pressure on the coat. However, the insoluble coating covering the passageway is unaffected by the fluid and the swelling pressure generated inside the system leads to development of a weak point in the coat at the junction of the insoluble coat and the permeable polymer. Hence, the coat ruptures and releases the trimipraine and optionally one or more additional active agents to the surrounding environment.

The multi-component compositions of the present invention may also be provided as a programmed drug delivery formulation that provides an immediate release of a trimipramine and optionally one or more additional active agents and a pulsed release of the trimipramine and optionally one or more additional active agents, the delay preferably being independent of gastric emptying time. The formulation provides a pulsatile plasma level time profile as defined above, with spaced pulses of the agent that typically are independent of the gastric emptying time.

At least one timed release component generally is present in the form of a core comprising trimipramine, one or more pharmaceutical excipients that swell upon exposure to aqueous medium, and optionally other pharmaceutically acceptable excipients, wherein the core is coated with a water insoluble and water impermeable coating.

A passageway may be drilled in the coat and covered with a band or a plug of a polymer composition that is soluble or swellable in the gastrointestinal fluids and whose solubility may be pH-dependent or pH-independent, preferably pH-independent. Upon erosion or dissolution of the soluble polymer the passageway is exposed and the fluid from the surrounding environment enters the system, causing it to swell and exert a pressure on the coat. The coat then ruptures to release the contents of the core.

Alternatively, the core may be coated with a polymer composition that is insoluble but permeable to water, and the passageway may be coated with a water-insoluble pH-independent polymer. The water entering the core through the permeable membrane causes the core to swell and the swelling exerts a pressure on the coat. However, the insoluble coating covering the passageway is unaffected by the fluid and the swelling pressure generated inside the system leads to development of a weak point in the coat at the junction of the insoluble coat and the permeable polymer. Hence, the coat ruptures and releases the trimipraine and optionally one or more additional active agents to the surrounding environment. A portion of the trimipramine and optionally one or more additional active agents is thereby released from the core after a delay.

The immediate release component may be present in the form of granules, pellets, beads, or tablets, or it may be present as an immediate release coat covering at least a part or whole of the pulsed release component. Alternatively, the immediate release component may be provided by mixing it with the water insoluble, water impermeable polymer, and using the mixture thus obtained to coat the pulsed release core.

The multi-component compositions of the invention may also be provided as formulations comprising a mixture of IR, sustained release and/or pulsed release vehicles (e.g. granules, powders, pellets, beads, suspensions, solutions, microspheres, seeds or combinations thereof). The IR, sustained release and/or pulsed release vehicles may be combined in any suitable dosage form, such as a capsule or capulet.

Such a formulation may contain an IR vehicle containing from about 20 to about 80% of the trimipramine contained in the formulation and a sustained and/or pulsed release vehicle containing from about 20 to about 80% of the trimipramine contained in the formulation.

The IR vehicle preferably is in the form of a powder, solution, suspension, granules or pellets of trimipramine. The sustained and/or pulsed release vehicle may be in the form of the immediate release vehicle mixed with a release-retarding material. As described in more detail hereinafter, the release-retarding material may be in the form of a matrix and/or coating (e.g. a waxy matrix or polymer coating). Alternatively, the sustained and/or pulsed release vehicle may be trimipramine formulated as a matrix, osmotic pump or microsphere.

The multi-component compositions of the invention may further be provided as a core of trimipramine coated with a release-retarding material, which is further coated with an IR component of trimipramine. The release-retarding material may be any suitable material as described in more detail hereinafter (e.g. a compression coating). The IR component of trimipramine may be a polymer coating comprising trimipramine.

The types of suitable multi-component formulations described above are illustrative and not limiting. Suitable pharmaceutically acceptable excipients which may be used to make the multi-component formulations described above, and in the remainder of this specification, are set out below.

The formulations described herein for the compositions of the invention are designed primarily for oral administration. Suitable oral dosage forms include, but are not limited to capsules, tablets, liquids, powders, granules, suspensions, matrices, microspheres, seeds, pellets and/or beads of the foregoing formulations. Combinations of these dosage forms may also be used in the invention. For example, an oral dosage form containing trimipramine may be in the form of microtablets enclosed inside a capsule, e.g. a hydroxypropylmethylcellulose (HPMC) capsule or a gelatin capsule. Any suitable gelatin capsule may be used, for example the hard gelatin capsule known as CAPSUGEL.

The compositions of the invention may be diffusion controlled formulations. By the term “diffusion controlled formulations”, we include formulations in which diffusion of dissolved trimipramine from the formulation has a significant role in the rate of controlled release of trimipramine from that formulation. However, dissolution processes may also be involved. Typical diffusion controlled formulations include so-called “reservoir systems”, in which a core of trimipramine is coated with a polymer (typically a water-insoluble polymer), and so-called “matrix systems”, in which the trimipramine is dispersed throughout a matrix (e.g. a swellable matrix), which may optionally be coated. In either system, flow and egress of the dissolved drug is controlled so as to achieve one or more of the release profiles defined above.

The compositions of the invention may be based on matrix technology. In this technology, trimipramine is embedded in an excipient that makes a non-disintegrating core called a matrix. Diffusion of (dissolved) trimipramine occurs through the core.

Preferably, the sustained release OD compositions of the invention are formulated so there is at least some time-delay before significant plasma concentrations of trimipramine are achieved. Such compositions may avoid an initial burst of trimipramine, or may be formulated so that release of trimipramine in a particular part of the gastrointestinal tract (e.g. the stomach) is retarded. This may be useful for minimizing any adverse event profiles associated with trimipramine.

Enteric coated formulations, which may protect the stomach against any irritant effects of trimipramine, might also be desirable. Such formulations can be coated with a composition that is non-toxic and includes a pharmaceutically acceptable enteric polymer, which is predominantly soluble in the intestinal fluid, but substantially insoluble in the gastric juices.

Typically, the compositions of the invention extend the trimipramine release, e.g. by several hours, compared to trimipramine release in the known IR dosage form.

The compositions of the invention may comprise a release-retarding material. The release-retarding material can be, for example, in the form of a matrix or a coating. The compositions of the invention may comprise, for example, a particle of trimipramine that is combined with a release-retarding material. The release-retarding material is typically a material that permits release of trimipramine at a sustained rate in an aqueous medium. The release-retarding material can be selectively chosen so as to achieve, in combination with the other stated properties, a desired release rate.

Release-retarding materials may be hydrophilic and/or hydrophobic polymers and/or materials. Suitable release-retarding materials include but are not limited to acrylic polymers, alkylcellulose, shellac, zein, hydrogenated vegetable oil, hydrogenated caster oil, and combinations comprising one or more of the foregoing materials. The compositions of the invention may contain between about 1% and about 80% (by weight) of the release-retarding material.

Suitable acrylic polymers include, for example, acrylic and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, and combinations comprising one or more of the foregoing polymers.

Suitable alkylcelluloses include, for example, ethylcellulose. Those skilled in the art will appreciate that other cellulosic polymers, including other alkyl cellulosic polymers, can be substituted for part or all of the ethylcellulose.

Other suitable hydrophobic materials are typically water-insoluble and may have a melting point of from about 30° C. to about 200° C., preferably from about 45° C. to about 90° C. The hydrophobic material may include neutral or synthetic waxes, fatty alcohols (such as lauryl, myristyl, stearyl, cetyl or preferably cetostearyl alcohol), fatty acids, including fatty acid esters, fatty acid glycerides (mono-, di-, and tri-glycerides), hydrogenated fats, hydrocarbons, hardened oils or fats (e.g. hardened rapeseed oil, caster oil, beef tallow, palm oil, soya bean oil) waxes, stearic acid, stearic acid, stearyl alcohol, polyethylene glycol, hydrophobic and hydrophilic materials having hydrocarbon backbones, and combinations comprising one or more of the foregoing materials.

Suitable waxes include beeswax, glycowax, castor wax, carnauba wax and wax-like substances, e.g. materials which are normally solid at room temperature and have a melting point of from about 30° C. to about 100° C., and combinations comprising two or more of the foregoing waxes.

The release-retarding material also may comprise digestible, long chain (e.g., C₈-C₅₀, preferably C₁₂-C₄₀), substituted or unsubstitued hydrocarbons, such as fatty acids, fatty alcohols, glyceryl esters of fatty acids, mineral and vegetable oils, waxes, and combinations comprising one or more of the foregoing materials. Hydrocarbons having a melting point of from about 25° C. to about 90° C. may be used. The compositions of the invention may contain up to about 60% by weight of at least one digestible, long chain hydrocarbon and/or up to 60% by weight of at least one polyalkylene glycol.

The release-retarding material also may comprise polylactic acid, polyglycolic acid, or a co-polymer of lactic acid and glycolic acid. The release-retarding material optionally includes other additives such as an erosion-promoting agent (e.g. starch and gums) and/or a semi-permeable polymer.

Release-modifying agents, which affect the release properties of the composition, may optionally be used in the compositions of the invention. The release-modifying agent may, for example, function as a pore-former. Typically, a pore-former creates channels which facilitate (e.g., accelerate) drug release. The pore former can be organic or inorganic, and may include materials that can be dissolved, extracted or leached from the coating in the environment of use. The pore-former can comprise one or more hydrophilic polymers, such as hydroxypropylmethylcellulose, lactose, metal stearates (e.g. alkali metal stearates such as magnesium stearate), polycarbonates (linear polyesters of carbonic acid in which carbonate groups reoccur in the polymer chain), and combinations comprising two or more of the foregoing release-modifying agents.

The release-retarding material can also include an exit means comprising at least one passageway, orifice, or the like. The passageway can have any shape, such as round, triangular, square or elliptical. Such exit means may be used in osmotic pump formulations and pulsed release formulations, which are described in more detail herein.

In addition to the above ingredients, the compositions of the invention may also contain suitable quantities of other materials, e.g. diluents, lubricants, binders, granulating aids, colorants, flavorants and glidants that are conventional in the pharmaceutical art.

Examples of suitable lubricants include stearic acid, magnesium stearate, glyceryl behenate, talc, mineral oil (in PEG). Examples of suitable binders include water-soluble polymers, such as modified starch, gelatine, polyvinylpyrrolidone, polyvinyl alcohol, etc. Examples of suitable fillers include lactose, microcrystalline cellulose. An example of a glidant is silicon dioxide.

The compositions of the invention may include one or more substrates comprising trimipramine. Such substrates may be coated with a sustained and/or pulsed and/or prolonged release coating comprising a release-retarding material. Such compositions may be used in a multiparticulate system, such as beads, ion-exchange resin beads, spheroids, microspheres, seeds, pellets, matrices, granules, and other multiparticulate systems in order to obtain the desired controlled release of trimipramine. The multiparticulate system can be presented in a capsule or other suitable unit dosage form, such as a tablet or a sachet.

In certain cases, more than one multiparticulate system may be used, each exhibiting different characteristics, such as pH dependence of release, time for release in various media (e.g. acid, base simulated intestinal fluid), release in vivo, size and composition.

In some cases, excipients to encourage spheronization may be used together with the active ingredient to form spheroids. Microcrystalline cellulose and hydrous lactose impalpable are examples of such spheronizing agents. Additionally (or alternatively), the spheroids may contain a water insoluble polymer, preferably an acrylic polymer, an acrylic copolymer, such as a methacrylic acid-ethyl acrylate copolymer, or ethyl cellulose. In such a formulation, any sustained release coating present may include a water insoluble material such as a wax, either alone or in admixture with a fatty alcohol, or shellac or zein.

Spheroids or beads, coated with an active ingredient may be prepared, for example, by dissolving the trimipramine in water and then spraying the solution onto a substrate such as sugar spheres. Optionally, additional ingredients may be added prior to coating the beads in order to assist the active ingredient binding to the substrates, and/or to colour the solution, etc. The resulting substrate-active material may be overcoated with a barrier material, to separate the trimipramine from the next coat of material, e.g. a release-retarding material. The barrier material may be a material comprising hydroxypropyl methylcellulose. However, any film-former known in the art may be used. Preferably, the barrier material increases stability during processing and/or shelf-life, without affecting the dissolution rate of the final product.

In order to achieve the desired release characteristics, trimipramine may be coated with an amount of release-retarding material sufficient to obtain a weight gain level from about 1 to about 80% (e.g. from about 2 to about 40%), although more or less release-retarding material may be used depending, for example, on the desired release-rate. Moreover, there may be more than one release-retarding material used in the coating, as well as various other pharmaceutical excipients.

The release-retarding material may be in the form of a film coating comprising a dispersion of a hydrophobic polymer. Solvents typically used for application of the release-retarding coating include pharmaceutically acceptable solvents, such as water, alcohols (e.g. methanol or ethanol), methylene chloride, and combinations comprising one or more of the foregoing solvents.

The in vivo and/or in vitro release profile of the compositions of the invention may be altered, for example optimised, by using more than one release-retarding material, by varying the thickness of the release-retarding material, by changing the particular release-retarding material used, by altering the relative amounts of release-retarding material, by altering the manner in which any plasticizer present is added, by varying the amount of plasticizer relative to retardant material, by the inclusion of additional ingredients or excipients, by altering the method of manufacture, or by combinations of the foregoing.

In addition to or instead of being present in a matrix, the release-retarding agent can be in the form of a coating. Optionally, a core can be coated, or a gelatine capsule can be further coated, with a sustained and/or pulsed and/or prolonged release coating such as those described herein. The coatings may include a sufficient amount of a hydrophobic material to increase the weight of the dosage from about 1 to about 80% (e.g. from about 2 to about 40%), although the coating can increase the weight of the dosage form by a larger percent depending on the desired release rate, among other factors.

The compositions of the invention preferably release trimipramine in a multi-component and/or prolonged manner when ingested and exposed to gastric fluids, and then to intestinal fluids. The controlled release profile of the formulations may be altered, for example, by varying the amount of release-retarding agent, e.g. hydrophobic material, by varying the amount any plasticizer present relative to hydrophobic material, by the inclusion of additional ingredients or excipients, by altering the method of manufacture, or combinations of the foregoing.

The compositions of the invention may be prepared in such a way that, substantially all of trimipramine is present in amorphous form. The term “amorphous” is intended to mean consisting of disordered arrangements of molecules which do not possess a distinguishable crystal lattice. A typical process for forming a composition comprising amorphous trimipramine comprises mixing trimipramine with water and a pharmaceutically acceptable polymeric carrier and drying the mixture to form a composition comprising amorphous trimipramine and the polymeric carrier.

Suitable pharmaceutically acceptable polymeric carriers include, for example, hydroxypropyl cellulose, methyl cellulose, carboxymethyl cellulose, sodium carboxymethyl cellulose, cellulose acetate phthalate, cellulose acetate butyrate, hydroxyethyl cellulose, ethyl cellulose, polyvinyl alcohol, polypropylene, dextran, dextrins, hydroxypropyl-beta-cyclodextrin, chitosan, lactic/glycolid copolymers, polyorthoester, polyanhydrate, polyvinyl chloride, polyvinyl acetate, ethylene vinyl acetate, lectins, carbopols, silicon elastomers, polyacrylic polymers, maltodextrins, lactose, fructose, inositol, trehalose, maltose, raffinose, polyvinylpyrrolidone (PVP), polyethylene glycol (PEG), and alpha-, beta-, and gamma-cyclodextrins, and combinations of the foregoing carriers.

Preferred polymeric carriers are one or more of polyvinylpyrrolidone, hydroxypropylmethyl cellulose, hydroxypropyl cellulose, methyl cellulose, block copolymers of ethylene oxide and propylene oxide, and polyethylene glycol. The polyvinylpyrrolidone (PVP) typically has an average molecular weight of from about 2,500 to about 3,000,000, for example from about 10,000 to about 450,000.

The polymeric carrier is preferably (i) miscible with both trimipramine free base and its pharmaceutically acceptable salts (especially the hydrochloride salt), (ii) capable of keeping the salt in a homogeneous noncrystalline solid state dispersion after the water has been removed by evaporation, (iii) chemically inert with respect trimipramine and (iv) at least partially water soluble, and more preferably is fully water soluble.

Trimipramine, the polymeric carrier, and water may be combined in any order. Typically, they are combined in a manner so as to form a solution of trimipramine and the polymeric carrier. In forming a solution of the polymeric carrier and water, heating the solution is not generally necessary at lower concentrations but is preferred at higher concentrations, provided that the temperature does not result in decomposition or degradation of any materials. It is preferred to add trimipramine after dissolving the polymeric carrier in water, suitably at from about 25 to about 100° C., for example from about 45 to about 80° C., in order to form a clear solution.

The ratio of trimipramine to the polymeric carrier can be varied depending, for example, on the precise release profile required. Typical weight ratios of polymeric carrier to trimipramine range from about 100:1 to about 0.5:1, preferably from about 50:1 to about 1:1, such as from about 20:1 to about 2:1 (e.g. about 5:1).

Upon formation of the (preferably clear) solution, the process proceeds by recovering the water to form a solid state dispersion of the trimipramine in the polymeric carrier. Any method of removal of the water which provides a homogeneous solid state dispersion can be used, suitable methods including evaporation under vacuum or spray drying. Methods of evaporation under vacuum include rotary evaporation, static vacuum drying and the combination thereof. One skilled in the art of pharmaceutical formulations can readily determine a reasonable temperature at which water can be removed, provided the temperature is not so high as to cause degradation or decomposition of the materials. Typically, evaporation occurs at from about 25° C. to about 100° C. Evaporation of water should provide a solid state dispersion which is homogeneous and substantially free of water. By substantially free it is meant that the solid state dispersion typically contains less than 20% by weight of residual water, preferably less than 10%, more preferably less than 5%, most preferably less than 1%.

Any suitable pharmaceutically acceptable excipient can be added to the compositions of the invention. Examples of pharmaceutically acceptable excipients include diluents, trimipramine vehicles, binders, disintegrants, glidants, sweeteners, compression aids, colouring agents, flavoring agents, suspending agents, dispersing agents, film formers, printing inks, lubricants and/or preservatives. These excipients may be used alone or in any combination.

The pharmaceutical composition may be formulated by conventional methods of admixture such as blending, filling, granulation and compressing. These agents may be utilized in a conventional manner.

Excipients may be added for numerous reasons, for example to facilitate manufacture, enhance stability, control release, enhance product characteristics, enhance bioavailability, enhance patient acceptability and combinations thereof.

Exemplary binders, which may be used to help to hold the dosage form together, include polyvinyl pyrrolidone, hydroxypropyl cellulose, hydroxypropyl methylcellulose, methylcellulose, hydroxyethyl cellulose, sugars, and combinations thereof. Disintegrants (such as croscarmellose sodium) expand when wet causing a tablet to break apart. Lubricants typically aid in the processing of powder materials. Exemplary lubricants include calcium stearate, glycerol behenate, magnesium stearate, mineral oil, polyethylene glycol, sodium stearylfumarate, stearic acid, talc, vegetable oil, zinc stearate, and combinations thereof. An example of a glidant is silicon dioxide.

The formulations described herein may contain a filler, such as a water insoluble or water soluble filler, or combinations thereof. Typical water insoluble fillers include silicon dioxide, titanium dioxide, talc, alumina, starch, kaolin, polacrilin potassium, powdered cellulose, microcrystalline cellulose, and combinations thereof. Typical water-soluble fillers include water soluble sugars and sugar alcohols, preferably lactose, glucose, fructose, sucrose, mannose, dextrose, galactose, the corresponding sugar alcohols and other sugar alcohols, such as mannitol, sorbitol, xylitol, and combinations thereof.

Trimipramine and any optional additives may be prepared as subunits or as pellets, for example by a melt pelletization technique. In this technique, the trimipramine in finely divided form is combined with a binder and other optional inert ingredients, and thereafter the mixture is pelletized, e.g. by mechanically working the mixture in a high shear mixer to form the pellets. By the term “pellets” we include pellets, granules, spheres and beads. Thereafter, the pellets can be sieved in order to obtain pellets of the requisite size.

The binder material may also be in particulate form and typically has a melting point above about 40° C. Suitable binder substances include hydrogenated castor oil, hydrogenated vegetable oil, other hydrogenated fats, fatty alcohols, fatty acid esters, fatty acid glycerides, and combinations thereof.

Oral dosage forms may be prepared to include an effective amount of subunits containing trimipramine and optionally other active agents in the form of multiparticles or multipellets within a capsule. For example, a plurality of multiparticulates may be placed in a gelatin capsule in an amount sufficient to provide one or more of the release profiles as defined above.

Subunits (e.g. in the form of multiparticulates) may be compressed into an oral tablet using conventional tableting equipment using standard techniques. The tablet formulation may include excipients such as, for example, an inert diluent (e.g. lactose) granulating and disintegrating agents (e.g. a cornstarch), binding agents (e.g. starch) and lubricating agents (e.g. magnesium stearate).

Alternatively, subunits containing trimipramine and optionally containing additional active agents may be subjected to an extrusion process, the resulting extrudate then being shaped into tablets by methods known in the art. The diameter of the extruder aperture or exit port can be adjusted to vary the thickness of the extruded strands. Furthermore, the exit part of the extruder may have any suitable shape, for example round, oblong or rectangular. The exiting strands can be reduced to particles using any suitable method, for example with a hot wire cutter or a guillotine.

A melt-extruded multiparticulate system can be, for example, in the form of granules, spheroids, pellets, or the like, depending upon the extruder exit orifice. The terms “melt-extruded multiparticulate(s)” and “melt-extruded multiparticulate system(s)” and “melt-extruded particles” are used interchangeably herein and typically include a plurality of subunits, preferably of similar size and/or shape. The melt-extruded multiparticulates are typically from about 0.1 to about 12 mm in length and from about 0.1 to about 5 mm in diameter. In addition, the melt-extruded multiparticulates can be any geometrical shape within this size range. Alternatively, the extrudate can simply be cut into desired lengths and divided into unit doses of trim ipramine without the need of a pelletization step.

Many of the oral dosage forms described herein contain trimipramine and optionally additional active agents in the form of particles. Such particles may be compressed into a tablet, present in a core element of a coated dosage form, such as a taste masked dosage form, a press coated dosage form, or an enteric coated dosage form, or may be contained in a capsule, osmotic pump dosage form, or other dosage form.

For particles (e.g. powder particles) present in the core element of a coated dosage form, the particles may have a particle size of from about 1 μm to about 250 μm, preferably from about 25 μm to about 200 μm, more preferably from about 35 μm to about 150 μm. The core element typically has a particle size distribution with a median of about 100 μm.

Another parameter to consider is the shape of the particles and/or any core element. For example, particle/core shape can influence the coverage and stability of any coating that may be used. Both the crystallinity of trimipramine and the aspect ratio of the particles are related to particle/core shape. If the trimipramine of the coated dosage has a crystalline morphology, sharp angles on the crystal can cause weaknesses (e.g. stress points) in the coat possibly leading to premature release of trimipramine from the dosage form. Furthermore, areas of thin coating are susceptible to breaking and cracking and hence less effective for sustained release and taste masking. This potential problem may be offset somewhat by the particles/core having a relatively low aspect ratio. The aspect ratio is a measure of the length to breadth. For example, a low aspect ratio of about 1 would be a box or sphere. Crystals with a high aspect ratio are more pointed with needle-like crystals. Crystals with a high aspect ratio may result in a relatively thin coat at the crystal needle tips leading to a more rapid release rate of trimipramine than is preferred. A low aspect ratio spherical shape of the particle is advantageous for both solubility of the coat and to increase the chance of all the trimipramine contained in the formulation being released. Therefore, it is most preferable that the aspect ratio is less than about 3, more preferably less than about 2, and most preferably approximately 1 providing a substantially rounded shape. This may be achieved, for example, by spheronisation.

Inconsistencies in size and shape can lead to inconsistent coating. Where the particles containing trimipramine are of different size and shape, polymeric coating materials such as ethyl cellulose may deposit differently on each particle. Therefore it is preferable for coated dosage forms that most if not all particles of the dosage form have substantially the same size and shape so that the coating process is better controlled and maintained.

The compositions described herein may be coated with a coating material. The coating typically comprises from about 0 to about 90% by weight of the composition. The coating material typically includes a polymer, preferably a film-forming polymer, for example, methyl cellulose, ethyl cellulose, hydroxypropyl cellulose, hydroxypropyl methyl cellulose, hydroxybutyl methyl cellulose, cellulose acetate, cellulose propionate, cellulose acetate propionate, cellulose acetate butyrate, cellulose acetate phthalate, carboxymethyl cellulose, cellulose triacetate, cellulose sulphate sodium salt, poly(methyl methacrylate), poly(ethyl methacrylate), poly(butyl methacrylate), poly(isobutyl methacrylate), poly(hexyl methacrylate), poly(phenyl methacrylate), poly(methyl acrylate), poly(isopropyl acrylate), poly(isobutyl acrylate), poly(octadecyl acrylate), high or low density, polyethylene, polypropylene, poly(ethyleneglycol), poly(ethylene oxide), poly(ethylene terephthalate), poly(vinyl alcohol), poly(vinyl isobutyl ether), poly(vinyl acetate), poly(vinyl chloride), polyvinyl pyrrolidone, and combinations thereof.

The coating material may be water soluble or water insoluble. For certain application such a taste-masking, it is preferable to use a water insoluble polymer. Suitable water insoluble polymers include ethyl cellulose or dispersions of ethyl cellulose, acrylic and/or methacrylic ester polymers, cellulose acetates, butyrates or propionates or copolymers of acrylates or methacrylates having a low quaternary ammonium content, and combinations of the foregoing polymers.

Preferred hydrophobic or water insoluble polymers for use in the compositions of the invention include, for example, methacrylic acid esters, ethyl cellulose, cellulose acetate, polyvinyl alcohol-maleic anhydride copolymers, β-pinene polymers, glyceryl esters of wood resins, and combinations of the foregoing.

The coating may also include one or more monomeric materials such as sugars (e.g. lactose, sucrose, fructose and mannitol), salts (e.g. sodium chloride and potassium chloride) and organic acids (e.g. fumaric acid, succinic acid, tartaric acid and lactic acid). The coating may also include a filler such as described earlier herein.

The coating composition may include additives which improve the physical properties of the coating film. For example, the coating composition may comprise a plasticizer. For example, because ethyl cellulose has a relatively high glass transition temperature and does not form flexible films under normal coating conditions, it may be advantageous to add plasticizer to the ethyl cellulose before using it as a coating material. Generally, the amount of plasticizer included in a coating solution is based on the concentration of the polymer, typically ranging from 0 to about 50% by weight of the coating composition. Suitable concentrations of the plasticizer may be determined by routine experimentation.

Examples of plasticizers for ethyl cellulose and other celluloses include plasticizers such as dibutyl sebacate, diethyl phthalate, triethyl citrate, tributyl citrate, triacetin, acetylated monoglycerides, phthalate esters, castor oil, and combinations thereof.

Examples of plasticizers for acrylic polymers include citric acid esters such as triethyl citrate 21, tributyl citrate, dibutyl phthalate, 1,2-propylene glycol, polyethylene glycols, propylene glycol, diethyl phthalate, castor oil, triacetin, acetylated monoglycerides, phthalate esters, castor oil, and combinations thereof.

A typical coating comprises (a) a poorly water-permeable component such as an alkyl cellulose (e.g. ethylcellulose) such as AQUACOAT (a 30% solution) or SURELEASE (a 25% solution) and (b) a water-soluble component, e.g. an agent that can form channels through the poorly water-permeable component upon the hydration or dissolution of the soluble component.

Preferably, the water-soluble component (b) is a low molecular weight, polymeric material, e.g. hydroxyalkylcellulose, hydroxyalkyl(alkylcellulose), carboxymethylcellulose, or salts thereof. Particular examples of these water soluble polymeric materials include hydroxyethylcellulose, hydroxypropylcellulose, hydroxyethylmethylcellulose, hydroxypropylmethyl cellulose (e.g. METHOCEL), carboxymethylcellulose, sodium carboxymethyl cellulose, and combinations thereof. The water-soluble component (b) is preferably of relatively low molecular weight, preferably less than about 25,000, preferably less about 21,000.

In the coating, the weight ratio of the water soluble component (b) to the poorly water permeable portion (a) is typically from about 1:4 to about 2:1, such as from about 1:2 to about 1:1, for example about 2:3. The coating typically constitutes from about 1 to about 90% by weight, such as from about 2% to about 50%, for example from about 5 to about 30%, of the weight of the total composition.

Preferably, the coating may be a substantially continuous coat and substantially hole-free. This is particularly advantageous, for example, where the coating provides taste masking. The phrase “substantially continuous coating” is meant to include a coating, which retains a smooth and continuous appearance when magnified 1000 times under a scanning electron microscope and wherein no holes or breakage of the coating are evident. Typically, the coating is from about 0.005 to about 25 μm thick, preferably from about 0.05 to about 5 μm.

One or more of the coatings described herein may be used in the compositions of the subject invention. If two or more coatings are present, the coating material used for each coating may be the same or different.

Any suitable method may be used to apply the coating. Processes which may be used include simple or complex coacervation, interfacial polymerization, liquid drying, thermal and/or ionic gelation, spray drying, spray chilling, fluidized bed coating, pan coating and electrostatic deposition. A substantially continuous coating may be achieved, for example, by spray drying from a suspension or dispersion of trimipramine in a solution of the coating composition including a polymer in a solvent in a drying gas having a low dew point.

When a solvent is used to apply the coating, the solvent is preferably an organic solvent which is a good solvent for the coating material and a poor solvent for trimipramine. While trimipramine may partially dissolve in the solvent, it is preferred that the active ingredient will precipitate out of the solvent during the spray drying process more rapidly than the coating material. The solvent may be selected from alcohols such as methanol, ethanol, halogenated hydrocarbons such as dichloromethane (methylene chloride), hydrocarbons such as cyclohexane, and combinations thereof.

The concentration of polymer in the solvent will normally be less than about 75% by weight, typically from about 10 to about 30% by weight. After coating, the coated dosage forms are typically allowed to cure for from about 1 to about 2 hours at a temperature of from about 50° C. to about 60° C.

The dosage form (e.g. a tablet) can be prepared by various conventional mixing, comminution and fabrication techniques readily apparent to those skilled in the chemistry of drug formulations. Examples of such techniques are direct compression (using appropriate punches and dies fitted to a suitable rotary tableting press), injection or compression molding using suitable molds fitted to a compression unit, granulation followed by compression, and extrusion into a mold or to an extrudate to be cut into lengths.

When particles or tablets are made by direct compression, the addition of lubricants to the particles/tablets may be helpful and sometimes important to promote powder flow and to prevent capping of the particle (breaking off of a portion of the particle) when the pressure is relieved. Useful lubricants are sodium stearyl fumarate, magnesium stearate (typically in a concentration of from about 0.25 to about 3% by weight in the powder mix), and hydrogenated vegetable oil, for example hydrogenated and refined triglycerides of stearic and palmitic acids may be used at from about 1 to about 5% by weight in the powder mix. Additional excipients may be added as fillers, to enhance powder flowability and reduce adherence.

Oral dosage forms may be prepared by including an effective amount of melt-extruded subunits in the form of multiparticles within a capsule. For example, a plurality of the melt-extruded multiparticulates can be placed in a gelatin capsule in an amount sufficient to provide the desired release profile when administered orally. Alternatively, the composition may be in the form of microtablets enclosed inside a gelatin capsule. Microtablets typically have a size of from 0.5 to 7 mm in their largest dimension, such as from 1 to 6 mm, for example 3 to 4 mm.

A number of formulations are described below as having preferred components. It is to be understood that any of the components described as being used in one type of formulation may also be used in another type of formulation, even though such components may not be listed as being used in the other formulation. Moreover, the formulations described below may also contain any of the excipients described above, or indeed any of the excipients known in the art.

The compositions of the invention may be in the form of a wax formulation. A wax formulation is a solid dosage form comprising the trimipramine in a waxy matrix.

The wax material used in the composition of the invention may be, for example, an amorphous wax, an anionic wax, an anionic emulsifying wax, a bleached wax, a carnauba wax, a cetyl esters wax, a beeswax, a castor wax, an emulsifying wax such as a cationic emulsifying wax, a cetrimide emulsifying wax, or a nonionic emulsifying wax, a glycerol behenate, a microcrystalline wax, a nonionic wax, a paraffin, a petroleum wax, a spermaceti wax, a white wax, and combinations of one or more of the foregoing waxes.

Acetyl ester wax suitable for use in the invention typically has a molecular weight of from about 470 to about 490, and is a mixture containing primarily esters of saturated fatty alcohols and saturated fatty acids. A wax matrix suitable for use in the compositions of the invention contains carnauba wax and no other waxy material. Another suitable wax matrix includes carnauba wax and glycerol behenates. The wax matrices suitable for use in the invention may be used with or without a coating.

The wax material may be used in the range of from about 30 to about 95%, preferably from about 40 to about 85%, more preferably from about 45 to about 80%, most preferably about 50% to about 75% by weight of the total weight of the matrix material. The remainder of the matrix material is typically trimipramine, although other optional components (e.g. fatty acid soaps, see below) may also be present. When a combination of waxes is used, the component waxes can be used in any suitable ratio. For example, if a combination of carnauba wax and glyceryl behenate is used, the relative amounts of each wax typically is from about 99 to 60 parts carnauba wax (for example from 99 to about 85 parts) and from about 1 to about 40 parts glyceryl behenate (for example from 1 to about 15 parts). In formulations that have a combination of carnauba wax and castor wax, the relative amounts of each wax typically is from about 99 to 60 parts carnauba wax (for example from 99 to about 85 parts) and from about 1 to about 40 parts castor wax (for example from 1 to about 15 parts). When carnauba wax, glyceryl behenate, and castor wax are present, the carnauba wax typically comprises at least about 85% of the waxy material present, the balance being made up of a combination of glyceryl behenate and castor wax.

Fatty acids and fatty acid soaps may be present in the waxy dosage form. In some cases, the fatty acids and/or fatty acid soaps can replace a portion of the wax material. These optional fatty acids and fatty acid soaps can be those that are generally used in the pharmaceutical industry as tableting lubricants. Such fatty acids and fatty acid soaps include solid fatty acids (for example fatty acids having from about 16 to about 22 carbon atoms), the alkaline earth metal salts thereof, (particularly the magnesium and calcium salts) and combinations of the foregoing. For example, the fatty acid can be stearic acid. The optional fatty acids and fatty soaps, when present, are typically used in amounts up to about 10% of the total weight of the matrix material, such as from about 1 to about 9%, for example from about 2 to about 8% or from about 3 to about 6% of the total weight of the matrix material.

To prepare the wax formulation, the wax or waxes may be melted and used to granulate trimipramine using melt granulation techniques. The granulate may be allowed to cool and then be milled to a proper size. Advantageously, the granulate is milled to an average particle size of about 75 microns to about 850 microns, preferably about 150 microns to about 425 microns. The milled granulate may be mixed with optional processing aids. The processing aids include, for example, hydrophobic colloidal silicon dioxide. Hydrophobic silicon dioxide may typically be used in amounts of less than or equal to about 0.5% by weight of the matrix material, but individual formulations can be varied as required. The blend of the waxy granulate and the processing aids, if any, may be compressed and then optionally coated.

The wax formulation may be formulated into any suitable dosage form, for example, coated (for example, with a functional coating composition or a non-function related coating composition) or uncoated tablets, compressed pellets contained in capsules, or loose powder or powder filled capsules.

When the coating composition is a functional coating composition, it typically comprises a water insoluble component and a water soluble component. When the coating composition is a non-functional coating composition, it typically comprises a water soluble component, preferably in the absence of a water insoluble component. The coating composition may comprise pharmaceutically acceptable dyes, pigments, or mixtures thereof.

As described in more detail below, the compositions of the invention may comprise one or more active agents in addition to trimipramine. Therefore, the wax formulation may also include an active agent in addition to trimipramine in the matrix.

The wax formulations described herein may be made by hot melting a waxy material to form a melt and granulating trimipramine with the melt to form a granulate. The granulate is typically then milled and compressed to form a matrix. The method may further comprise blending the granulate with a processing aid prior to compressing the granulate to form the matrix. The method may further comprise coating the matrix with a functional and/or a non-functional coating.

The compositions of the invention may be in the form of press-coat formulations. Such formulations comprise a core composition containing trimipramine with a coating composition press-coated on the core. The core composition typically comprises a waxy material containing trimipramine. The coating composition typically comprises a hydrophilic polymer and optionally trimipramine.

The waxy material of the core composition is typically a hydrophobic waxy material capable of providing controlled release of trimipramine. Such waxy materials may be, for example, carnauba wax, tribehenin, fatty alcohols (particularly those having 12-24 carbon atoms, such as lauryl alcohol, myristyl alcohol, stearyl alcohol, palmityl alcohol, etc.), fatty acids (particularly those having 12-24 carbon atoms, such as lauric acid, myristic acid, stearic acid, palmitic acid, etc), polyethylenes, castor wax, C₁₆₋₃₀ fatty acid triglycerides, beeswax, and combinations of one or more of the foregoing waxes.

The hydrophilic polymer of the coating composition is typically chosen so as to aid controlled release of trimipramine. An example of such a hydrophilic polymer is a film-forming polymer, such as a hydrophilic cellulose polymer, in particular a hydroxyalkyl cellulose polymer. Examples of such hydroxyalkyl cellulose polymers include hydroxyethylcellulose (HEC), hydroxypropylcellulose (HPC), hydroxypropylmethylcellulose (HMPC), hydroxypropylethylcellulose (HPEC), hydroxypropylpropylcellulose (HPPC), hydroxypropylbutylcellulose (HPBC), and combinations of one or more of the foregoing polymers.

Both the core composition and the coating composition may independently include a filler, such as a water soluble or insoluble filler, or a mixture thereof. Examples of water insoluble fillers include talc and calcium salts such as a calcium phosphate, e.g. a dicalcium phosphate. If there is a filler in the coating composition, it can be the same or different as the filler in the core composition, if any. For example, the core composition may include a water-soluble filler while the coating composition may include a water-insoluble filler.

Optional excipients can also be present in the core composition and/or the coating composition. Such excipients include lubricants (such as talc and magnesium stearate), glidants (such as fumed or colloidal silica), pH modifiers (such as acids, bases and buffer systems), pharmaceutically useful processing aids, and combinations of one or more of the foregoing excipients. Excipients in the compositions can be the same or different as those in the core compositions.

In order to form the press-coat formulations, the core composition components (trimipramine, waxy material, and optional excipients) are typically blended together and compressed into suitable cores. The blending can take place in a suitable order of addition. The cores may be blended by starting with the smallest volume component and then successively adding the larger volume components. An alternative process is to melt the wax and to blend trimipramine and optional excipients into the melted wax. Alternatively, trimipramine, wax and any optional excipients can be blended together and then subjected to a temperature at which the wax will melt. Once cooled, the solidified mass can be milled into granules for compaction into cores.

Typically, the core composition is press-coated with the coating composition to form a tablet. The tablet may be further coated with optional additional coatings. The additional coatings can be pH-dependent or pH-independent, aesthetic or functional, and can contain trimipramine or a different active agent.

If trimipramine is present in the coating composition, the molar ratio of trimipramine in the core composition to trimipramine in the coating composition is from about 500:1 to about 1:10, such as from about 100:1 to about 1:5, e.g. from about 10:1 to about 1:1.

A preferred press-coat formulation comprises a core composition comprising trimipramine coated with a coating composition comprising hydroxypropylmethyl cellulose (HPMC). The core composition optionally comprises one or more waxy materials, e.g. carnauba wax and the coating composition optionally comprises trimipramine (e.g. in the immediate release portion of the multi-component compositions of the invention). Such press coat formulations may be prepared by press-coating the coating composition onto the core composition.

The compositions of the invention may be formulated using osmotic pump technology. Osmotic pump technology uses osmotic pressure to deliver trimipramine at a controlled rate. Osmotic pump dosage formulations typically include a semi-permeable membrane surrounding a core that contains at least two components, one component comprising trimipramine, the other comprising an osmotic push layer (an osmotically active expandable driving member), such as an osmotically active polymer. After the dosage form is swallowed, water enters the membrane at a rate primarily determined by the nature of the membrane. This causes the push layer to swell, releasing trimipramine at the desired and controlled rate through an exit means comprising a passageway or orifice (e.g. a laser-drilled hole) by the action of the osmotically active driving member.

The osmotic pump formulation typically comprises a semipermeable membrane, for example a capsule or tablet or other dosage form typically having an outer wall comprising a selectively semipermeable material. The selectively permeable material preferably has the following characteristics: (i) it does not adversely affect a host or animal, (ii) it is permeable to the passage of an external aqueous fluid, such as water or biological fluids while remaining essentially impermeable to the passage of trimipramine, (iii) it is substantially insoluble in body fluids, (iv) it is non-toxic, and (v) it is non-erodible in the environments to which it is subjected.

Representative materials for forming the selectively semipermeable wall include semipermeable homopolymers and copolymers. Suitable materials include, for example, cellulose esters, cellulose monoesters, cellulose diesters, cellulose triesters, cellulose ethers, cellulose ester-ethers, and combinations thereof. These cellulosic polymers have a degree of substitution (DS) on their anhydroglucose unit from greater than 0 to about 3. The “degree of substitution” is the average number of hydroxyl groups originally present on the anhydroglucose unit that have been replaced by a substituting group, or converted into another group. The anhydroglucose unit can be partially or completely substituted with semipermeable polymer forming groups such as acyl, alkanoyl, aroyl, alkenyl, alkoxy, halogen, carboalkyl, alkylcarbamate, alkylcarbonate, alkylsulfonate and alkylsulfamate.

Other selectively semipermeable materials include, for example, cellulose acylate, cellulose diacylate, cellulose triacylate, cellulose acetate, cellulose diacetate, cellulose triacetate, mono-, di- and tri-cellulose alkanylates, mono-, di- and tri-alkenylates, mono-, di- and tri-aroylates, and combinations of the foregoing materials. Exemplary polymers include cellulose acetate having a DS of 1.8 to 2.3 and an acetyl content of about 32 to about 40%, cellulose diacetate having a DS of 1 to 2 and acetyl content of about 21 to about 35%, cellulose triacetate having a DS of 2 to 3 and an acetyl content of about 34 to about 45%. Other examples of cellulosic polymers include cellulose propionate having a DS of 1.8 and a propionyl content of about 38.5%, cellulose acetate propionate having an acetyl content of about 1.5 to about 7% and a propionyl content of about 39 to about 42%, cellulose acetate propionate having an acetyl content of about 2.5% to about 3%, an average propionyl content of about 39 to about 45% and a hydroxyl content of about 2.8% to about 5.4%. Still further exemplary cellulosic polymers include cellulose acetate butyrate having a DS of 1.8, an acetyl content of about 13 to about 15% and a butyryl content of about 34% to about 39%, cellulose acetate butyrate having an acetyl content of about 2 to about 29.5%, a butyryl content of about 17 to about 53%, and a hydroxyl content of about 0.5% to about 4.7%. Yet further examples of suitable cellulosic polymers include cellulose triacylates have a DS of 2.9 to 3 such as cellulose trivalerate, cellulose trilaurate, cellulose tripalmitate, cellulose trioctanoate, and cellulose tripropionate, cellulose diesters having a DS of 2.2 to 2.6 such as cellulose disuccinate, cellulose dipalmitate, cellulose dioctanoate, cellulose dicarpylate, mixed cellulose esters such as cellulose acetate valerate, cellulose acetate succinate, cellulose propionate succinate, cellulose acetate octanoate, cellulose valerate palmitate, cellulose acetate heptonate, and combinations of the foregoing cellulosic polymers polymers.

Other potentially suitable semipermeable polymers include, for example, acetaldehyde dimethyl cellulose acetate, cellulose acetate ethylcarbamate, cellulose acetate methylcarbamate, cellulose dimethylaminoacetate, semipermeable polyamides, semipermeable polyurethanes, semipermeable polysulfanes, semipermeable sulfonated polystyrenes, cross-linked selectively semipermeable polymers formed by the coprecipitation of a polyanion and a polycation, semipermeable silicon rubbers, semipermeable polystyrene derivatives, semipermeable poly(sodium styrenesulfonate), semipermeable poly(vinylbenzyltrimethyl)ammonium chloride polymers, and combinations comprising any of the foregoing polymers, including combinations with one or more of the selectively permeable materials listed in the preceding paragraph.

The osmotically expandable driving member (or osmotic push layer) of the osmotic pump dosage form is typically a swellable and expandable inner layer. The materials suitable for forming the osmotic push layer, include polymeric materials and/or polymeric materials blended with osmotic agents, both of which typically interact with water or a biological fluid, absorb the fluid, and swell or expand to an equilibrium state in the presence of the fluid without dissolving. Preferably, the polymer should exhibit the ability to retain a significant fraction of absorbed fluid in the polymer molecular structure. Such polymers may be gel polymers that can swell or expand to a very high degree, for example exhibiting from about 2 to about 50-fold volume increase.

Suitable swellable, hydrophilic polymers, also known as osmopolymers, can be non-cross-linked or lightly cross-linked. The cross-links can be covalent or ionic bonds with the polymer. The polymer may be of plant, animal or synthetic origin. Polymeric materials useful for the present purpose include poly(hydroxyalkyl methacrylate) having a molecular weight of from about 5,000 to about 5,000,000, poly(vinylpyrrolidone) having a molecular weight of from about 10,000 to about 360,000, anionic and cationic hydrogels, poly(electrolyte) complexes, poly(vinyl alcohol) having a low acetate residual, a swellable mixture of agar and carboxymethyl cellulose, a swellable composition comprising methyl cellulose mixed with a sparingly crosslinked agar, a water-swellable copolymer produced by a dispersion of finely divided copolymer of maleic anhydride with styrene, ethylene, propylene, or isobutylene, water swellable polymers of N-vinyl lactams, and combinations of the foregoing polymers.

Other gelable, fluid absorbing and retaining polymers useful for forming the osmotic push layer include pectins having a molecular weight ranging from about 30,000 to about 300,000, polysaccharides such as agar, acacia, karaya, tragacanth, algins and guar, poly (carboxylic acids) and their salt derivatives, polyacrylamides, water-swellable indene maleic anhydridge polymers, polyacrylic acid having a molecular weight of from about 80,000 to about 200,000, polyethylene oxide polymers having a molecular weight of from about 100,000 to about 5,000,000 (but may be higher), starch graft copolymers, polyanion and polycation exchange polymers, starch-polyacrylonitrile copolymers, acrylate polymers with water absorbability of from about 100 to about 600 times their original weight, diesters of polyglucan, a mixture of cross-linked polyvinyl alcohol and poly(N-vinyl-2-pyrrolidone), zein (available as prolamine), poly(ethylene glycol) having a molecular weight of from about 4,000 to about 100,000, and combinations of the foregoing polymers.

The osmotically expandable driving layer of the osmotic pump dosage form may further contain an osmotically effective compound (osmagent) that can be used neat or blended homogeneously or heterogeneously with the swellable polymer discussed above. Such osmagents are typically osmotically effective solutes that are soluble in the fluid absorbed into the swellable polymer, and exhibit an osmotic pressure gradient across the semipermeable wall against an exterior fluid.

Suitable osmagents include, for example, solid compounds such as magnesium sulfate, magnesium chloride, sodium chloride, lithium chloride, potassium sulfate, sodium sulfate, mannitol, urea, sorbital, inositol, sucrose, glucose, and combinations thereof. The osmotic pressure of the osmagents is typically from about 0 to about 500 atm, but may be higher.

The swellable, expandable polymer of the osmotically expandable driving layer, in addition to providing a driving source for delivering trimipramine from the dosage form, may also function as a supporting matrix for an osmotically effective compound (or osmagent). The osmotic compound may be homogeneously or heterogeneously blended with the polymer to yield the desired expandable wall or expandable pocket. A typical osmotic pump dosage form may comprise from about 20 to about 90% by weight of polymer and from about 80 to about 10% by weight of osmotic compound, preferably from about 35 to about 75% by weight of polymer and from about 65 to about 25% by weight of osmotic compound based on the total weight of the formulation.

The trimipramine in the osmotic pump dosage form may be formulated in any suitable manner, for example as a thermo-responsive formulation in which trimipramine is dispersed in a thermo-responsive composition. Alternatively, the osmotic pump dosage form may contain a thermo-responsive element comprising a thermo-responsive composition at the interface of the osmotic push layer and trimipramine composition. Representative thermo-responsive compositions (including their melting points in parentheses) are cocoa butter (32° C.-34° C.), cocoa butter and 2% beeswax (35° C.-37° C.), propylene glycol monostearate and distearate (32° C.-35° C.), hydrogenated oils such as hydrogenated vegetable oil) (36° C.-37.5°, 80% hydrogenated vegetable oil and 20% sorbitan monopalmitate (39° C.-39.5° C.), 80% hydrogenated vegetable oil and 20% polysorbate 60, (36° C.-37° C.), 77.5% hydrogenated vegetable oil, 20% sorbitan trioleate, 2.5% beeswax and 5.0% distilled water, (37° C.-38° C.), mono-di, and triglycerides of acids having from 8-22 carbon atoms including saturated and unsaturated acids such as palmitic, stearic, oleic, lineolic and archidonic; triglycerides of saturated fatty acids with mono- and diglycerdies (34° C.-35.5° C.), propylene glycol mono- and distearates (33° C.-34° C.), partially hydrogenated cottonseed oil (35° C.-39° C.), block copolymers of polyoxyalkylene and propylene glycol, block copolymers of 1,2-butylene oxide and ethylene oxide, block copolymers of propylene oxide and ethylene oxide, hardened fatty alcohols and fats (33° C.-36° C.), hexadienol and hydrous lanolin triethanolamine glyceryl monostearate (38° C.), eutectic mixtures of mono-, di-, and triglycerides (35° C.-39° C.), WITEPSOL#15, triglyceride of saturated vegetable fatty acid with monoglycerides (33.5° C.-35.5° C.), WITEPSOL H32 free of hydroxyl groups (31° C.-33° C.), WITEPSOL W25 having a saponification value of 225-240 (33.5° C.-35.5° C.), WITEPSOL E75 having a saponification value of 220-230 (37° C.-39° C.), a polyalkylene glycol such as polyethylene glycol 1000, a linear polymer of ethylene oxide (38° C.-41° C.), polyethylene glycol 1500 (38° C.-41° C.), polyethylene glycol monostearate (39° C.-42.5° C.), 33% polyethylene glycol 1500, 47% polyethylene glycol 6000 and 20% distilled water (39° C.-41° C.), 30% polyethylene glycol 1500, 40% polyethylene glycol 4000 and 30% polyethylene glycol 400, (33° C.-38° C.), mixtures of mono-, di- and triglycerides of saturated fatty acids having 11 to 17 carbon atoms, (33° C.-35° C.), and mixtures of the foregoing.

The thermo-responsive compositions, including thermo-responsive carriers, are thought to be useful for storing trimipramine in a solid composition at a temperature of about 20° C. to about 33° C., maintaining an immiscible boundary at the swelling composition interface, and for dispensing the agent in a flowable composition at a temperature greater than about 33° C. and preferably from about 33° C. to about 40° C.

When the trimipramine containing thermo-responsive formulations described above are used, the integrity of the semi-permeable membrane which is also present in such osmotic pump formulations is preferably not compromised (e.g. melted or eroded) by the presence of the thermo-responsive formulations.

Trimipramine in the osmotic pump dosage form may be formulated by any suitable techniques known in the art, for example by wet granulation or fluid bed granulation, as described in more detail below.

Firstly, trimipramine and the ingredients comprising the trimipramine layer are blended using an organic solvent, such as isopropyl alcohol-ethylene dichloride 80:20 v/v (volume:volume) as the granulation fluid. Other granulating fluid such as denatured alcohol 100% may be used for this purpose. The ingredients forming the trim ipramine layer are individually passed through a screen such as a 40-mesh screen and then thoroughly blended in a mixer. Next, other ingredients comprising the trimipramine layer are dissolved in a portion of the granulation fluid. Then the latter prepared wet blend is slowly added to the trimipramine blend with continual mixing in the blender. The granulating fluid is added until a wet blend is produced, which wet mass then is forced through a screen such as a 20-mesh screen and onto oven trays. The blend is dried for about 18 to about 24 hours at about 30° C. to about 50° C. The dry granules are sized then with a screen such as a 20-mesh screen. Next, a lubricant is passed through a screen such as an 80-mesh screen and added to the dry granule blend. The mixture is put into milling jars and mixed on a jar mill for about 1 to about 15 minutes. The push layer may also be made by the same wet granulation techniques. The compositions are pressed into their individual layers in a KILIAN press-layer press.

Another manufacturing process that can be used for providing the trimipramine layer and the osmotically expandable driving layer comprises blending the powered ingredients for each layer independently in a fluid bed granulator. After the powered ingredients are dry blended in the granulator, a granulating fluid (e.g. poly(vinyl-pyrrolidone) in water, denatured alcohol, 95:5 ethyl alcohol/water, or blends of ethanol and water) is sprayed onto the powders. Optionally, the ingredients can be dissolved or suspended in the granulating fluid. The coated powders are then typically dried in a granulator. This process granulates the ingredients present therein while adding the granulating fluid. After the granules are dried, a lubricant such as stearic acid or magnesium stearate is added to the granulator. The granules for each separate layer may then be pressed then in the manner described above for the wet granulation method.

The osmotic push trimipramine formulation and osmotic push layer of the osmotic push dosage form may also be manufactured by mixing trimipramine with composition forming ingredients and pressing the composition into a solid lamina. In a further alternative method of manufacture, trimipramine, any other composition-forming ingredients and a solvent are typically mixed into a solid, or a semisolid, by methods such as ballmilling, calendaring, stirring or rollmilling, and then pressed into a preselected layer forming shape. Next, a layer of composition comprising an osmopolymer and an optional osmagent are typically placed in contact with the layer comprising trimipramine. The layering of the first layer comprising trimipramine and the second layer comprising the osmopolymer and optional osmagent composition may be accomplished by using a conventional layer press technique.

The semipermeable wall can be applied by molding, spraying or dipping the pressed bilayer's shapes into wall forming materials. An air suspension coating procedure which includes suspending and tumbling the two layers in a current of air until the wall forming composition surrounds the layers may also be used to form the semi-permeable wall of the osmotic formulations.

The dispenser of the osmotic pump dosage form may be, for example, in the form of a hard or soft capsule. The capsule may also be osmotic.

The hard capsule may be composed of two parts, a cap and a body, which are typically fitted together after the body (which is generally larger than the cap) is filled with trimipramine. The hard capsule may be fitted together by slipping or telescoping the cap section over the body section, thus completely surrounding and encapsulating trimipramine.

The soft capsule of the osmotic pump dosage form may be a one-piece soft capsule. Typically, the soft capsule comprises a sealed construction encapsulating trimipramine. The capsule may be made by various processes, such as the plate process, the rotary die process, the reciprocating die process, and the continuous process.

Materials useful for forming the capsule of the osmotic pump dosage form may be commercially available materials including gelatin (typically having a viscosity of about 5 to about 30 millipoises and a bloom strength up to about 150 grams or gelatin having a bloom value of about 150 to about 250), a composition comprising gelatin, glycerine, water and titanium dioxide, a composition comprising gelatin, erythrosine, iron oxide and titanium dioxide, a composition comprising gelatin, glycerine, sorbitol, potassium sorbate and titanium dioxide, a composition comprising gelatin, acacia, glycerine, and water and combinations thereof. Commercially available gelatin capsules (e.g. CAPSUGEL) may also be used.

The semipermeable wall forming composition may be applied to the trimipramine containing component and/or to the exterior surface of the capsule in laminar arrangement by molding, forming, air spraying, dipping or brushing. Alternative techniques that can be used for applying the semipermeable wall include air suspension procedures and pan coating procedures. For example, an air suspension procedure includes suspending and tumbling the capsule arrangement in a current of air and a semipermeable wall forming composition until the wall surrounds and coats the capsule. The procedure can be repeated with a different semipermeable wall forming composition to form a semipermeable laminated wall.

Exemplary solvents suitable for manufacturing the semipermeable wall include inert inorganic and organic solvents that do not adversely harm the materials used in the osmotic pump formulations, e.g. the capsule wall, trimipramine, the thermo-responsive composition, the expandable member, or the final dispenser. Such solvents include aqueous solvents, alcohols, ketones, esters, ethers alipathics hydrocarbons, halogenated solvents, cycloaliphatics, aromatics, heterocyclic solvents, and combinations thereof. Particular solvents include acetone, diacetone alcohol, methanol, ethanol, isopropyl alcohol, butyl alcohol, methyl acetate, ethyl acetate, isopropyl acetate, n-butyl acetate, methyl isobutyl ketone, methyl propyl ketone, n-hexane, n-heptane, ethylene glycol monoethyl ether, ethylene glycol monoethyl acetate, methylene dichloride, ethylene dichloride, propylene dichloride, carbon tetrachloride, nitroethane, nitropropane, tetrachloroethane, ethyl ether, isopropyl ether, cyclohexane, cyloocatane, benzene, toluene, naphtha, 1,4-dioxane, tetrahydrofuran, water, and mixtures thereof such as acetone and water, acetone and methanol, acetone and ethyl alcohol, methylene dichloride and methanol, and ethylene dichloride, methanol, and combinations of the foregoing.

The exit means or hole in the osmotic pump formulations for releasing trimipramine may be produced during manufacture or in use. For example, the exit means or hole can be formed by mechanical or laser drilling, or by eroding an erodible element in the wall, such as a gelatine plug. The orifice can be a polymer inserted into the semipermeable wall, which polymer is a (micro)porous polymer which typically has at least one (micro)pore.

An example of a formulation for the controlled of trimipramine in the stomach and gastrointestinal tract is one in which trimipramine is dispersed in a polymeric matrix that is water-swellable rather than merely hydrophilic. Such water-swellable matrices typically also have an erosion rate that is substantially slower than their swelling rate, and release trimipramine primarily by diffusion.

The rate of diffusion of trimipramine from the matrix can be modified by varying numerous characteristics of the formulation. For example, the rate of diffusion of trimipramine can be slowed by increasing trimipramine particle size, by the choice of polymer used in the matrix, and/or by the choice of molecular weight of the polymer. The matrix is typically a relatively high molecular weight polymer that swells upon ingestion, preferably to a size that is at least about twice its unswelled volume, and that promotes gastric retention. Upon swelling, the matrix may convert over a prolonged period of time (such as from about 1 to about 48 hours, e.g. from about 2 to about 24 hours or from about 3 to about 12 hours) from a glassy or crystalline polymer to a polymer this rubbery in consistency.

Typically, penetrating fluid causes release of trimipramine in a gradual (i.e., sustained) or pulsed manner by the process of solution diffusion, i.e. dissolution of trim ipramine in the penetrating fluid and diffusion of the dissolved drug backed out of the matrix.

Typically, the matrix itself is solid prior to administration, and once administered, remains undissolved in (i.e. is not eroded by) the gastric fluid for a period of time sufficient to permit the majority of trimipramine to be released in a controlled manner (as defined by the release profiles described above) by solution diffusion. Therefore, the rate-limiting factor in the release of trimipramine is believed to be controlled diffusion of trimipramine from the matrix rather than erosion, dissolving or chemical decomposition of the matrix.

The water-swellable polymer which forms the matrix is a polymer that is non-toxic, that swells in a dimensionally unrestricted manner upon absorption of water (and/or other fluids) and that provides for sustained release of incorporated trimipramine. Examples of suitable polymers include, for example, cellulose polymers and their derivatives (such as hydroxyethylcellulose, hydroxypropylcellulose, carboxymethylcellulose, and microcrystalline cellulose), polysaccharides and their derivatives, polyalkylene oxides, polyethylene glycols, chitosan, poly(vinyl alcohol), polysaccharide gums, maleic anhydride copolymers, poly(vinyl pyrrolidone), starch and starch-based polymers, poly(2-ethyl-2-oxazoline), poly(ethyleneimine), polyurethane hydrogels, crosslinked polyacrylic acids and their derivatives, copolymers of the foregoing polymers, including block copolymers and grafted polymers (e.g. PLURONIC and TECTONIC, which are polyethylene oxide-polypropylene oxide block copolymers) and mixtures thereof.

As used herein, unless otherwise stated, the terms “cellulose” and “cellulosic” denote a linear polymer of anhydroglucose. Suitable cellulosic polymers include, for example, alkyl-substituted cellulosic polymers that ultimately dissolve in the gastrointestinal (GI) tract in a predictably pulsed manner. Specific examples are methylcellulose, hydroxymethyl-cellulose, hydroxyethylcellulose, hydroxypropylcellulose, hydroxypropylmethylcellulose, and carboxymethylcellulose. The viscosity of suitable alkyl-substituted cellulosic polymers is typically from about 100 to about 110,000 centipoise as a 2% aqueous solution at 20° C. or from about 1,000 to about 4,000 centipoise as a 1% aqueous solution at 20° C. Exemplary alkyl-substituted celluloses are hydroxyethylcellulose and hydroxypropylmethylcellulose. A specific example of a hydroxyethylcellulose is NATRASOL 250HX NF.

Suitable polyalkylene oxides are those having the properties described above for alkyl-substituted cellulose polymers. An example of a polyalkylene oxide is poly(ethylene oxide) (PEO), which term is used herein to denote a linear polymer of unsubstituted ethylene oxide. Suitable PEO polymers typically have molecular weights of greater than about 4,000,000, preferably from about 4,500,000 to about 10,000,000, more preferably about from 5,000,000 to about 8,000,000. Preferred polyethylene oxides are those with a weight-average molecular weight ranging from about 1×10⁶ to about 1×10⁷, preferably from about 9×10⁵ to about 8×10⁶. Suitable PEOs typically have a viscosity of from about 50 to about 2,000,000 centipoise as a 2% aqueous solution at 20° C. Two specific example of PEOs are POLYOX NF, grade WSR Coagulant, molecular weight 5 million, and grade WSR 303, molecular weight 7 million.

Examples of suitable polysaccharide gums are natural and modified (semi-synthetic) polysaccharide gums such as dextran, xanthan gum, gellan gum, welan gum and rhamsan gum.

Suitable crosslinked polyacrylic acids include those whose properties are the same as or similar to those described above for alkyl-substituted cellulose and polyalkylene oxide polymers. Typically, such crosslinked polyacrylic acids have a viscosity of about 4,000 to about 40,000 centipoise as a 1% aqueous solution at 25° C. Three specific examples are CARBOPOL NF grades 971P, 974P and 934P. Further examples include polymers known as WATER LOCK, which are starch/acrylates/acrylamide copolymers.

As mentioned above, the hydrophilicity and water-swellability of the polymers discussed above cause trimipramine-containing matrices to swell in size in the gastric cavity due to ingress of water and/or other fluids. This swelling promotes retention of the matrices in the stomach during the fed phase. The hydrophilicity and water-swellability also cause the matrices to become slippery, which provides resistance to peristalsis and further promotes their retention in the stomach.

The release rate of trimipramine from the matrix is primarily dependent upon the rate of water absorption and the rate at which trimipramine dissolves and diffuses from the swollen polymer, which in turn is related to the solubility and dissolution rate of trimipramine, trimipramine particle size and trimipramine concentration in the matrix. Also, because these matrix-forming polymers typically dissolve very slowly in gastric fluid, the matrix maintains its physical integrity over at least a substantial period of time, typically for at least 70 or 80% of the dosing period, and in many cases at least 90% and even over 100% of the dosing period. Generally, the particles then slowly dissolve or decompose. Complete dissolution or decomposition may not occur until 24 hours or more after administration, although in many cases, complete dissolution or decomposition will occur within 10 to 24 hours after the dosing period.

The swellable matrix dosage forms may include additives that impart a small degree of hydrophobic character, to further retard the release rate of trimipramine into the gastric fluid. Examples of such release rate retardants are glyceryl monostearate, fatty acids and salts of fatty acids, (e.g. sodium myristate). Typically, the weight ratio of additive to trimipramine is in the range of from about 1:10 to about 10:1, for example from about 1:5 to about 5:1.

The amount of polymer relative to trimipramine may vary, depending on the precise nature of the desired release profile, its molecular weight, and excipients that may be present in the formulation. However, the amount of polymer will be sufficient so that the polymeric matrix will remain substantially intact until all of trimipramine is released. The term “substantially intact” is used herein to denote a polymeric matrix in which the polymer portion substantially retains its size and shape without deterioration due to becoming solubilized in the gastric fluid or due to breakage into fragments or small particles.

The water-swellable polymers can be used individually or in combination. Certain combinations will often provide a more controlled release of trimipramine than their components when used individually. Such combinations include cellulose-based polymers (e.g. hydroxyethyl cellulose or hydroxypropyl cellulose) or poly(ethylene oxide) combined with gums, (e.g. xanthan gum).

The benefits of the swellable matrix dosage form are typically achieved over a wide range of trimipramine loadings, for example weight ratios of trimipramine to polymer of from about 0.001:1 to about 10:1. Typical loadings (expressed in terms of the weight percent of trimipramine relative to trimipramine and polymer combined) are from about 0.001% to about 50%, preferably from about 0.01% to about 40%, such as from about 0.1% to about 30%, for example from about 1% to about 20%.

The swellable matrix formulations may find their greatest utility when administered to a subject who is in the digestive state (also referred to as the postprandial or “fed” mode). The postprandial mode is distinguishable from the interdigestive (or “fasting”) mode by distinct patterns of gastroduodenal motor activity, which determine the gastric retention or gastric transit time of the stomach contents.

Such localisation of trimipramine release in the stomach and small intestine reduces and/or prevents substantial colonic degradation, inactivation, or loss of bioavailability.

Juvenile and elderly patients often require dosage forms that are easy to swallow, for example to reduce the risk of choking upon administration, and/or to improve patient compliance. The compositions of the invention may be in the form of easily administerable dosage forms, making them more suitable for patient compliance. Such easily administerable formulations include, for example, sprinkle dosage forms, taste-masked liquid dosage forms, fast-dissolve dosage forms and chewable dosage forms.

It is to be understood that any of the easily administerable dosage forms described below may comprise any of the formulations described above in order to provide a composition which has one or more of the desired release profiles of trimipramine according to the subject invention.

An example of a chewable dosage form is a trimipramine-containing chewable tablet. Such a chewable tablet comprises a chewable base and optionally a sweetener. The chewable base typically comprises an excipient such as mannitol, sorbitol, lactose, or a combination thereof. The optional sweetener used in the chewable dosage form may be, for example, sucrose, liquid glucose, sorbitol, dextrose, isomalt, liquid maltitol, aspartame, lactose, or a combination thereof. In certain cases, the chewable base and the sweetener may be the same component. The chewable base and optional sweetener typically comprise about 50% to about 90% by weight of the total weight of the chewable dosage form.

The chewable dosage form may additionally contain preservatives, agents that retard and/or prevent adhesion to the oral cavity and crystallization of sugars, flavouring agents, souring agents, colouring agents, and combinations of one or more of the foregoing. Glycerin, lecithin, hydrogenated palm oil or glyceryl monostearate may be used as a protecting agent of crystallization of the sugars, typically in an amount of from about 0.01 to about 2% by weight of the total weight of the ingredients. Such protecting agents help to prevent adhesion to oral cavity and improve the soft property or chewability of the dosage form. Additionally or alternatively, isomalt or liquid maltitol may be used to enhance the chewing properties of the chewable dosage form.

The method for making the chewable dosage form comprising trimipramine described above is similar to the method used to make soft confectionary. Such a method typically involves the formation of a boiled sugar-corn syrup blend to which is added a frappe mixture. The boiled sugar-corn syrup blend may be prepared from sugar and corn syrup blended in parts by weight ratio of 90:10 to 10:90. This blend may be heated to temperatures above 120° C. to remove water and to form a molten mass. The frappe mixture may be prepared from gelatine, egg albumen, milk proteins such as casein, and vegetables proteins such as soy protein, and the like which are added to a gelatine solution and rapidly mixed at ambient temperature to form an aerated sponge like mass. The frappe mixture is then added to the molten candy base and mixed until homogenous, typically at temperatures between 60° C. to about 120° C. A matrix, tablet or other formulation containing trimipramine may then be added to the mix at a temperature of from about 60° C. to about 90° C., whereupon additional ingredients such as flavours, colourants, and preservatives may be added. The formulation is then typically cooled and formed to pieces of desired dimensions.

Fast-dissolving dosage forms may comprise microparticles and one or more effervescent agents, enabling the dosage forms to rapidly disintegrate in the mouth whilst providing adequate taste-masking. Alternatively, rapidly dissolving dosage forms may contain an active agent and a matrix that includes a nondirect compression filler and a lubricant. U.S. Pat. No. 5,178,878 and U.S. Pat. No. 6,221,392 provides teachings regarding fast-dissolve dosage forms.

Typical fast dissolve dosage forms for use in the subject invention include a mixture incorporating a water and/or saliva activated effervescent agent, a disintegration agent, and microparticles. The microparticles typically incorporate trimipramine together with a protective material substantially encompassing the trimipramine. The term “substantially encompassing” includes the meaning that the protective material substantially shields trimipramine from contact with the environment outside the microparticle. Thus, each microparticle may incorporate a discrete mass of trimipramine covered by a coating of the protective material, in which case the microparticle can be referred to as a “microcapsule” or a “microtablet”. Alternatively or additionally, each microparticle may have trimipramine dispersed or dissolved in a matrix of the protective material, optionally coated by a coating composition as described herein.

The mixture including the microparticles and an effervescent agent is typically present as a tablet of a size and shape adapted for direct oral administration to a patient. The tablet is substantially completely disintegrable upon exposure to water and/or saliva. The effervescent disintegration agent is present in an amount effective to aid disintegration of the tablet, and to provide a distinct sensation of effervescence when the tablet is placed in the mouth of a patient.

The effervescent sensation is typically not only pleasant to the patient but also tends to stimulate saliva production, thereby providing additional water to aid in further effervescent action. Thus, once the tablet is placed in the patient's mouth, it will generally disintegrate rapidly and substantially completely without any voluntary action by the patient. Thus, even if the patient does not chew the tablet, disintegration should proceed rapidly. Upon disintegration of the tablet, the microparticles are released and can be swallowed as a slurry or suspension of the microparticles. The microparticles are thus transferred to the patient's stomach for dissolution in the digestive tract and systemic distribution of the trimipramine.

The terms “effervescent agent” and “disintegration agent” includes compounds which evolve gas. Such agents may evolve gas by means of chemical reactions which take place upon their exposure to water and/or to saliva in the mouth. The bubble or gas generating reaction is most often the result of the reaction of a soluble acid source and an (alkali metal) carbonate source. The reaction of these two general classes of compounds produces carbon dioxide gas upon contact with the water in saliva.

Such saliva/water-activated materials should be kept in a generally anhydrous state with little or no absorbed moisture or in a stable hydrated form since exposure to water will prematurely disintegrate the tablet. For example, the dosage form may be stored in substantially air-tight packaging prior to administration.

The acid source may be any which is safe for human consumption and may generally include food acids, acid anhydrides and acid salts. Food acids include citric acid, tartaric acid, malic acid, fumaric acid, adipic acid, and succinic acids, etc. Because these acids are directly ingested, their overall solubility in water is less important than it would be if the formulations were intended to be dissolved in a glass of water. Acid anhydrides and acid salts of the above-described acids may also be used. Acid salts may include sodium, dihydrogen phosphate, disodium dihydrogen pyrophosphate, acid citrate salts and sodium acid sulfite.

The carbonate source includes dry solid carbonate and bicarbonate salts such as sodium bicarbonate, sodium carbonate, potassium bicarbonate and potassium carbonate, magnesium carbonate and sodium sesquicarbonate, sodium glycine carbonate, L-lysine carbonate, arginine carbonate, amorphous calcium carbonate, and combinations thereof.

While the effervescent disintegration agent is typically one which upon a reaction which forms carbon dioxide, this is not essential. Effervescent disintegration agents which evolve oxygen or other gasses which are safe for human patients may also be used.

Where the effervescent agent included two mutually reactive components, such as an acid source and a carbonate source, it is preferred that both components react substantially completely. Therefore, an equimolar ratio of acid and carbonate sources is preferred. For example, if the acid used is diprotic, then either twice the molar amount of a mono-reactive carbonate base, or an equal molar amount of a di-reactive base should be used for complete neutralization to be realized. However, the amount of either acid or carbonate source may exceed the amount of the other component. This may be useful to enhance taste and/or performance of a tablet containing an excess of either component. In such cases, it is acceptable that the additional amount of either component may remain unreacted.

The fast-dissolving dosage forms (e.g. tablets) typically contain an amount of effervescent disintegration agent effective to aid rapid and complete disintegration of the tablet when orally administered. By “rapid”, it is understood that the tablets should disintegrate in the mouth of a patient in less than 10 minutes, such as from about 15 seconds and about 7 minutes, for example from about 30 seconds and about 5 minutes. Disintegration time in the mouth can be measured by observing the disintegration time of the tablet in water at about 37° C. The tablet is immersed in the water without forcible agitation. The disintegration time is the time from immersion for substantially complete dispersion of the tablet as determined by visual observation. As used herein, the term “complete disintegration” of the tablet does not require dissolution or disintegration of the microcapsules or other discrete inclusions.

In order to achieve such disintegration, the amount of effervescent agent or disintegration agent typically used in the fast-dissolve dosage forms is from about 5% to about 50% by weight of the final composition, preferably from about 15% to about 40% by weight, more preferably about 20% to about 30% by weight.

The tablets described above can be manufactured by well-known tableting procedures.

As mentioned above, each microparticle typically incorporates trimipramine in conjunction with a protective material. The microparticle may be provided as a microcapsule, microtablet or as a matrix-type microparticle. Microcapsules may incorporate a discrete mass of trimipramine surrounded by a discrete, separately observable coating of the protective material. Conversely, in a matrix-type particle, trimipramine is dissolved, suspended or otherwise dispersed throughout the protective material. Certain microparticles may include attributes of both microcapsules and matrix-type particles. For example, a microparticle may incorporate a core incorporating a dispersion of trimipramine in a first protective material and a coating of a second protective material, which may be the same as or different from the first protective material surrounding the core. Alternatively, a microparticle may incorporate a core consisting essentially of trimipramine and a coating incorporating the protective material, the coating itself having some trimipramine dispersed within it. The microparticles typically have a mean diameter of from about 75 to about 600 microns, preferably from about 150 to about 500 microns, for example from about 200 to about 450 microns. The microparticles may be from about 200 to about 30 mesh (US standard size), for example from about 100 to about 35 mesh.

The protective materials suitable for use in the fast dissolve dosage forms described above typically include polymers which are conventionally utilized in the formation of microparticles such as matrix-type microparticles, microtablets and microcapsules. Among these are cellulosic materials such as naturally occurring cellulose, synthetic cellulose derivatives, acrylic polymers and vinyl polymers. Other simple polymers including may also be used, such as proteinaceous materials (e.g. gelatine, polypeptides) and natural and synthetic shellacs and waxes. Protective polymers may also include ethylcellulose, methylcellulose, carboxymethyl cellulose and acrylic resin material.

When a coating is used in the above fast dissolve dosage forms, it typically comprises at least about 5% by weight based on the total weight of the resulting particles, preferably at least about 10% by weight. The upper limit of protective coating material used is generally less critical. In certain embodiments it is possible to use a coating that is greater than 100 percent of weight of the core, providing a relatively thick coating. However, the amount of coating material should not be so great that it impedes the release of a therapeutically effective amount trimipramine before defecation of the dosage form.

An example of a fast-dissolve dosage form is a hard, compressed, rapidly dissolvable dosage form adapted for direct oral dosing. Such a dosage form typically includes trimipramine, often in the form of a protected particle, and a matrix. The matrix typically includes a filler and a lubricant, although it may include other additional ingredients. The dosage form is adapted to rapidly dissolve in the mouth of a patient, yet it has a friability of about 2% or less when tested according to the USP. Generally, the dosage form will also have a hardness of at least about 1.5 or 2.0 kP. Not only does the dosage form dissolve quickly, it does so in a way that provides a positive organoleptic sensation to the patient. In particular, the dosage form dissolves with a minimum of unpleasant grit, which is tactilely very inconsistent with organoleptic sensation of the dosage form.

The filler typically comprises a non-direct compression filler. Exemplary fillers include, for example, nondirect compression sugars and sugar alcohols. Such sugars and sugar alcohols include dextrose, mannitol, sorbitol, lactose, and sucrose. Dextrose, for example, can exist as either a direct compression sugar, i.e., a sugar that has been modified to increase its compressibility or a nondirect compression sugar. The percentage of filler is typically in the range of from about 25 to about 98% by weight of the microparticles, preferably from about 50 to about 95%, for example from about 60 to about 90%.

In the fast-dissolve dosage forms discussed above, a relatively high proportion of lubricant is typically used. Lubricants, and in particular, hydrophobic lubricants such as magnesium stearate, may be used in an amount of from about 0.25 to about 5% by weight of the formulation, preferably from about 1 to about 3% by weight, for example from about 1.5 to about 2% by weight. Despite the use of this relatively high percentage weight of lubricant, the formulations typically exhibit excellent compressibility, hardness, and rapid dissolution within the mouth.

Hydrophobic lubricants include, for example, alkaline earth metal stearates, stearic acid, mineral and vegetable oils, glyceryl behenate, sodium stearyl fumarate, and combinations thereof. Hydrophilic lubricants may be also be used.

The hard, compressed fast-dissolve dosage forms typically have a hardness of at least about 1.5 kP and are designed to dissolve spontaneously and rapidly in the mouth of a patient in less than about 90 seconds to thereby liberate the particles. Preferably the dosage form will dissolve in less than about 60 seconds and even more preferably in about 30 to about 45 seconds. This measure of hardness is based on the use of small tablets of less than about 0.25 inches in diameter. A hardness of at least about 2.0 kP is preferred for larger tablets. Direct compression techniques are preferred for the formation of these tablets.

Sprinkle dosage forms are another form of easily administered formulations that may be used in the compositions of the invention. Sprinkle dosage forms typically comprise trimipramine in the form of pellets, granules, microtablets or microcapsules, optionally having functional or non-functional coatings. In use, the patient or caregiver can sprinkle the particulate/pelletized dose into drink or onto soft food. A sprinkle dosage form may comprise particles having a mean diameter of from about 10 to about 100 ^{c}_{c}m in their major dimension, for example from about 50 to 70 ^{c}_{c}m.

An example of a sprinkle dosage form is an easily openable capsule enclosing a plurality of trimipramine-containing micropellets. Each of the micropellets typically comprises a seed coated with a first coating mixture of trimipramine and polyvinylpyrrolidone and a second coating mixture of from about 90 to about 70% by weight of the mixture of a non-hydrophilic polymer (e.g. ethyl cellulose) and from about 10 to about 30% by weight of the mixture of a hydrophilic polymer (e.g. hydroxypropyl methyl cellulose). For example, the second coating mixture may comprise about 3 parts ethylcellulose to about 1 part hydroxypropylcellulose. The weight of the second coating mixture is about 5-10% of the weight of the micropellets before the second coating is applied. Optionally, the second coating contains trimipramine.

The polyvinylpyrrolidone used in the first coating typically has a molecular weight of from about 30,000 to about 50,000, e.g. about 40,000. The seed of the sprinkle dosage form may be a sugar seed and have a mesh size of 60/80.

Taste-masked dosage forms are another form of easily administered formulations that may be used in the compositions of the invention. The taste-masked dosage form may be liquid or solid.

A solid taste masked dosage form typically comprises a core element comprising trimipramine and a coating material surrounding the core element. The core element comprising trimipramine is typically in the form of a (micro)particle, (micro)tablet, (micro)capsule, amorphous solid, pellet, granule, powder or a matrix. The core element may include carriers or excipients, fillers, flavouring agents, stabilizing agents and/or colourants in addition to trimipramine.

The taste-masked dosage form typically includes from about 50 to about 99% by weight, preferably from about 65 to about 95% by weight, for example from about 80 to about 90% by weight of the trim ipramine-containing core element, based on the total weight of the dosage form. The taste-masked dosage form typically includes from about 1 to about 50% by weight, preferably from about 5 to about 35% by weight, for example from about 10 to about 20% by weight of the coating material surrounding the core element, based on the total weight of the dosage form.

The core element typically includes from about 20 to about 90% by weight of a supplementary component selected from waxes, water insoluble polymers, enteric polymers, and partially water soluble polymers, other suitable pharmaceutical excipients, and combinations thereof.

The core element optionally include carriers or excipients, fillers, flavouring agents, stabilizing agents, colorants, and combinations thereof. Suitable fillers include, for example, insoluble materials such as silicon dioxide, titanium dioxide, talc, alumina, starch, kaolin, polacrilin potassium, powdered cellulose, and microcrystalline cellulose, and combinations comprising one or more of the foregoing fillers. Soluble fillers include, for example, mannitol, sucrose, lactose, dextrose, sodium chloride, sorbitol, and combinations comprising one or more of the foregoing fillers. The filler may be present in amounts of up to about 75% by weight based on the total weight of the dosage form.

The core element may be in the form of a powder, for example, having a particle size range of about 35 ^{c}_{c}m to about 125 ^{c}_{c}m. Such small particle size facilitates a substantially non-gritty feel in the mouth. Small particle size also minimizes break-up of the particles in the mouth, e.g. by the teeth. When in form of a powder, the taste masked dosage form may be administered directly into the mouth or mixed with a carrier such as water, or semi-liquid compositions such as syrups, yogurt, and the like. However, the taste-masked trimipramine may be provided in any suitable unit dosage form.

The coating material of the taste-masked formulation may take a form that provides a substantially continuous coating and provides taste masking. The coating may also provide controlled release of trimipramine. The polymer used in taste masked dosage form coating may be a water insoluble polymer such as, for example, ethyl cellulose. The coating material of the taste masked dosage form may further include a plasticizer.

A method of preparing taste-masked pharmaceutical formulations such as powdered formulations typically includes mixing a core element and a coating material in a diluent and spray drying the mixture to form a taste-masked formulation. Spray drying of the pharmaceutically active ingredient and polymer in the solvent typically involves spraying a stream of air into an atomized suspension, optionally in a drying chamber, so that solvent is caused to evaporate leaving trim ipramine coated with the polymer coating material.

For a solvent such as methylene chloride, the solvent concentration in the drying chamber is typically maintained at from about 40,000 to about 100,000 parts per million of organic solvent. The spray-drying process for such solvents may be conducted at a process temperature of about 5° C. to about 35° C. Spray drying of the dosage forms may be undertaken utilizing either rotary, pneumatic or pressure atomizers located in either a co-current or mixed-flow spray dryer or variations thereof. The drying gas may be heated or cooled to control the rate of drying. A temperature below the boiling point of the solvent may be used. Inlet temperatures may be from about 40 to about 120° C. and outlet temperatures from about 5° C. and 35° C.

The coat formation may be optimized to meet the needs of the material or application. Controlling the process parameters such as temperature, solvent concentration, spray dryer capacity, atomizing air pressure, droplet size, viscosity, total air pressure in the system and the solvent system, allows the formation of a range of coats, ranging from dense, continuous, non-porous coats through to more porous microcapsule/polymer matrices.

A post-treatment step may be used to remove any residual solvent. The post treatment may include a post drying step including drying the final product on a tray and/or at a bed temperature sufficient to remove excess solvent, but not degrade the trimipramine. Preferably the drying temperature is in the range of from about 35° C. to about 40° C. Once completed, the product may be collected by a suitable method, such as collection by sock filters or cyclone collection.

An exemplary chewable taste-masked dosage form comprises a microcapsule of about 10 ^{c}_{c}m to about 1.5 mm in diameter having a core comprising trimipramine and a polymer mixture coating having sufficient elasticity to withstand chewing. The polymeric mixture coating typically comprises from about 30 to about 70% by weight of a polymer that forms a polymeric film at temperatures of at least about 30° C. (e.g. ethylcellulose) and from about 30 to about 70% by weight of a copolymer that forms a polymeric film at temperatures less than about 25° C. The polymeric mixture coating is adapted so that the dosage form exhibits the release profiles discussed earlier in this specification.

The copolymer that forms a polymeric film at temperatures less than about 25° C. is typically a methacrylic acid ester copolymer (having, for example, a weight average molecular weight of about 800,000) or a styrene acrylate copolymer.

The core of the taste-masked trimipramine dosage form described above may comprise a diluent and/or a plasticizer. Suitable plasticizers, include, but are not limited to polyethylene glycol, triacetin, vinylpyrrolidone, diethyl phthalate, dibutylsebacate, a citric acid ester, and combinations thereof.

Solid taste-masked dosage forms (e.g. polymer coated trimipramine powder) may be reconstituted as suspensions in a liquid vehicle such as water before usage. This has the advantage that the reconstitutable solid taste-masked dosage forms typically have a longer shelf life than many liquid taste-masked dosage forms and the suspensions, once reconstituted, have adequate taste masking.

Trimipramine is a tricyclic antidepressant which imparts its clinical activity via its antagonistic effects on the monoamine neurotransmitters in the brain. In addition it displays specific antagonism of the dopamine D2 receptors and has been shown to have antipsychotic effects.

It is currently used for the treatment of all forms of Depression and has an indication for the treatment of primary or secondary Insomnia (www.fda.gov). The subject invention seeks to address the deficiencies of known trimipramine-containing formulations for the treatment of Depression and Insomnia by providing the orally deliverable pharmaceutical compositions described herein. However, these compositions may also be used in the treatment of numerous other medical indications in addition to Depression and Insomnia, as described in more detail below.

The subject invention provides the use of an orally deliverable pharmaceutical composition as defined in the claims for the treatment of a neurological and/or a psychiatric condition.

By the term “a neurological and/or a psychiatric condition”, we include all conditions deriving from a pathology of the nervous system. Particular examples of such conditions are described in more detail below.

The phrase “the treatment of a neurological and/or a psychiatric condition” is intended to include use for the acute, chronic and/or prophylactic treatment of neurological, neuropsychiatric, psychiatric and neurodegenerative disease.

Accordingly, there are numerous conditions which may be treated by administering or using the compositions of the invention. These include all Depressive disorders and symptoms, Primary and Secondary Insomnia, Schizophrenia, Bipolar Disorders, schizoaffective disorders, Anxiety disorders (including Generalised Anxiety disorder), obsessive compulsive disorder, Post Traumatic Stress Disorder, Personality Disorder and Borderline Personality Disorder, all types of dementia and cognitive impairment (e.g. alzheimers disease, mild cognitive impairment of the elderly etc.); psychiatric complications of stroke (including haemorrhagic and ischaemic and sequelae), epilepsy, transient ischaemic attacks, traumatic brain injury, Parkinsons disease, Huntingtons disease, amytrophic lateral sclerosis; neuropathic pain, idiopathic pain, all psychoses (such as degenerative Depression and catatonia), all addictions, (e.g. addiction to alcohol, nicotine and opiates), all eating disorders including bulimia and anorexia, affective disorders including ADHD (attention deficit hyperactivity disorder), all depressive disorders, personality disorders (including borderline personality disorders), sleep disorders (including jet lag and insomnia), Downs syndrome, meningitis, central nervous system vasculitis, leukodystrophies and adrenoleukodystrophies (including Alexander's disease, Canavan's disease, cerebrotendinous xanthomatosis, Krabbes and metachromatic LD), fatigue, hypoglycaemia, encephalopathy, (such as hepatic and septic encephalopathy), tumours of the brain and spinal cord (including primary tumours of glial, neuronal, schwann cell, pinealcyte, meningioma, melanoma, sarcoma, lymphoma and multiple systemic systemic malignancies which metasize), cerebellar degeneration and ataxias (e.g. Friedrich's ataxia, cerebellar cortical atazia, complicated cerebellar ataxia, which includes olivopontocerebellar degeneration, spinocerebellar disease, dentatorubral degeneration and autosomal dominant ataxias) vertigo, vestibular system damage, cochlear disorders such as tinnitus, nystagmus, peripheral neuropathy, (e.g. polyneuropathy, polyradiculopathy, motor neuronopathy, sensor neuronopathy, multiple mononeuropathy and plexopathies), metabolic bone diseases, osteoporosis, pulmonary disorders, (such as pulmonary edema, neurogenic pulmonary edema, bronchial asthma, adult respiratory distress syndrome (ARDS) and pulmonary cell death by apoptosis or necrosis), obesity and complications thereof, diabetes and prediabetes, and combinations thereof.

Compositions of the invention that are suitable for once nightly administration are believed to be particularly suitable for a combined treatment of insomnia and depression because they are thought to achieve the desired clinical effect at least in part overnight. For example, the compositions of the invention may be administered anytime during the evening before going to bed, such as from about 4 hours to immediately before going to bed.

For example, a single component sustained release OD composition may be administered daily before going to bed (e.g. at about 8 pm).

Optionally, a composition may be administered before going to bed that comprises an IR component which releases a first component of trimipramine and a pulsed and/or sustained release component which releases a second component of trimipramine overnight and/or in the morning (e.g. at about 8 am).

Alternatively, a composition may be administered before going to bed that comprises a sustained release component which releases a first component of trimipramine overnight and a pulsed release component which releases a second component of trimipramine in the morning. In addition, such a formulation may have significant utility if dosed in the morning (e.g. at about 8 am) as the second component of trimipramine release would coincide with the patient wishing to retire to bed for which further drug release would be advantageous.

As noted above, the compositions of the invention may comprise one or more active agents in addition to trimipramine.

For example, the compositions of the invention may comprise another atypical antipsychotic agent (e.g. olanzapine, quetiapine, risperidone, amisulpride, clozepine, chlorpromazine, or haloperidol decanoate), antiparkinsonian agents (e.g. L-DOPA, Dopamine Agonists), sedatives (e.g. a benzodiazepine sedative or non-barbituate sedative), anxiolytics (e.g. benzodiazepines such as lorazepam, chlordiazepoxide, oxazepam, clorazepate, diazepam, and alprazolam), antidepressants, and mood stabilisers (e.g. lamotrigine, lithium, valproate, carbamazepine, oxcarbazepine).

The antiparkinsonian agents may be used to treat the tardive dyskinesia associated with neuroleptic use. Also called “side-effect medication” antiparkinsonians are indicated when muscle side-effects of the atypical antipsychotics make patients uncomfortable. Antiparkinsonian agents are typically anticholinergic drugs, examples including benztropine mesylate, trihexyphenidyl, procyclidine, and amantadine.

Suitable antidepressents include tricyclic antidepressants (such as amitriptyline, imipramine, doxepin, and clomipramine), monoamine oxidase A or B inhibitors (such as phenelzine andtranylcypromine), tetracyclic antidepressants (e.g. maprotiline), and serotonin re-uptake inhibitors such as fluoxetine, cipramil, S-cipramil, paroxetine, and sertraline hydrochloride, serotonin and nor adrenaline reuptake inhibitors such as venlafaxine and duloxetine, nor adrenaline reuptake inhibitors such as reboxetine and viloxazine and all other classes of antidepressants.

Of course, the Trimipramine formulations described herein may be used for the treatment of numerous other conditions in addition to Insomnia and Depression. Such conditions may require treatment by different additional active agents (in addition to Trimipramine) than those described above in relation to the treatment of Insomnia and Depression.

The invention will now be illustrated by the following non-limiting Examples.

### EXAMPLE 1

### Trimipramine Compositions

100 mg direct compression (DC) controlled release tablets were manufactured as described below.

The ingredients set out in Tables 1-4 below were blended together in a tumble blender for 5 minutes. The blend was compressed on a rotary tabletting machine, using 8.5 mm diameter round n/c punches. The tablet breaking strength was 8.0 kp to 11.0 kp.

TABLE 1
Direct Compression Composition A (DC A)

| Batch # DJ1/57/20% K4M | % w/w | Tablet (mg) | Batch (g) |
|---|---|---|---|
| Trimipramine maleate | 46.5 | 139.5 | 23.25 |
| Hydroxypropyl methylcellulose K4M | 20.0 | 60.0 | 10.00 |
| Microcrystalline cellulose PH200 | 32.5 | 97.5 | 16.25 |
| Sodium stearyl fumarate | 1.0 | 3.0 | 0.50 |
| | 100.0 | 300.0 | 50.00 |

TABLE 2
Direct Compression Composition B (DC B)

| Batch # DJ1/58/30% K4M | % w/w | Tablet (mg) | Batch (g) |
|---|---|---|---|
| Trimipramine maleate | 46.5 | 139.5 | 23.25 |
| Hydroxypropyl methylcellulose K4M | 30.0 | 90.0 | 15.00 |
| Microcrystalline cellulose PH200 | 22.5 | 67.5 | 11.25 |
| Sodium stearyl fumarate | 1.0 | 3.0 | 0.50 |
| | 100.0 | 300.0 | 50.00 |

TABLE 3
Direct Compression Composition C (DC C)

| Batch # DJ1/59/40% K100 | % w/w | Tablet (mg) | Batch (g) |
|---|---|---|---|
| Trimipramine maleate | 46.5 | 139.5 | 23.25 |
| Hydroxypropyl methylcellulose K100 | 40.0 | 120.0 | 20.00 |
| Microcrystalline cellulose PH200 | 12.5 | 37.5 | 6.25 |
| Sodium stearyl fumarate | 1.0 | 3.0 | 0.50 |
| | 100.0 | 300.0 | 50.00 |

TABLE 4
Direct Compression Composition D (DC D)

| Batch # DJ1/62 30% K100 | % w/w | Tablet (mg) | Batch (g) |
|---|---|---|---|
| Trimipramine maleate | 46.5 | 139.5 | 83.75 |
| Hydroxypropyl methylcellulose K100 | 30.0 | 90.0 | 54.00 |
| Microcrystalline cellulose PH200 | 22.5 | 67.5 | 40.50 |
| Sodium stearyl fumarate | 1.0 | 3.0 | 1.80 |
| | 100.0 | 300.0 | 180.00 |

In the above tablets, the trimipramine maleate was obtained from Sigma Aldrich (USA). The methocel K100 & K4M ((hydroxypropyl methylcellulose 2208 (hypromellose)) was obtained from Colorcon Limit (UK). Avicel PH 200 (microcrystalline cellulose) was obtained from FMC BioPolymer (Ireland). Sodium Stearyl Fumarate, under the trademark PRUV, was obtained from JRS Pharma GMBH (Germany).

### EXAMPLE 2

### In Vitro Release Experiments

The release profiles of Trimipramine from the DC tablets described in Example 1 were studied in 0.1M HCl, as described in more detail below.

### Dissolution System

| | |
|---|---|
| Dissolution medium | 0.1M Hydrochloric Acid |
| Apparatus | USP II (Paddles) |
| Volume | 900 ml |
| Speed | 100 rpm |
| Temperature | 37° C. |

The 0.1M HCl was prepared by diluting 3.5 litres of 0.2M hydrochloric acid to 7 litres with purified water.

### Dissolution Procedure

A single tablet was tested at any one time, using a Varian dip probe coupler 02-101593-00. The probe had a 10 mm path length and was coupled to a Cary 50 UV spectrometer. A zero reading was taken in 0.1M HCl at 37° C. and sample readings taken automatically at 30 min intervals.

A single standard was used and absorbance measured at 37° C. A standard concentration was used, equivalent to approximately 100 mg in 1 litre trimipramine, made from trimipramine maleate using a conversion factor of 1.396.

Calculation:

$$\% Released = \frac{Sample Absorbance \times mg std ( in 900 ml )}{Standard Absorbance}$$

For composition C the following procedure was also used for each of 4 tablets:

Aliquots were taken from each dissolution vessel at the indicated intervals. The UV absorbance of each aliquot at 247 nm was measured against a blank solution of 0.1M HCl and calibrated against four reference standards of 0.015, 0.03, 0.09 and 0.12 mg/ml trimipramine (calculated from the maleate using a factor of 1.396). The % trimipramine dissolved is calculated using the calibration curve created from the reference standards:
p=purity of reference standard as % w/w (not required when the input batch of drug substance is used as the reference standard)
X=value obtained from graph in mg/ml

$$\% dissolved = \frac{X \times 900 \times p \times 100 \%}{Label claim ( mg ) \times 100}$$

### Dissolution Results

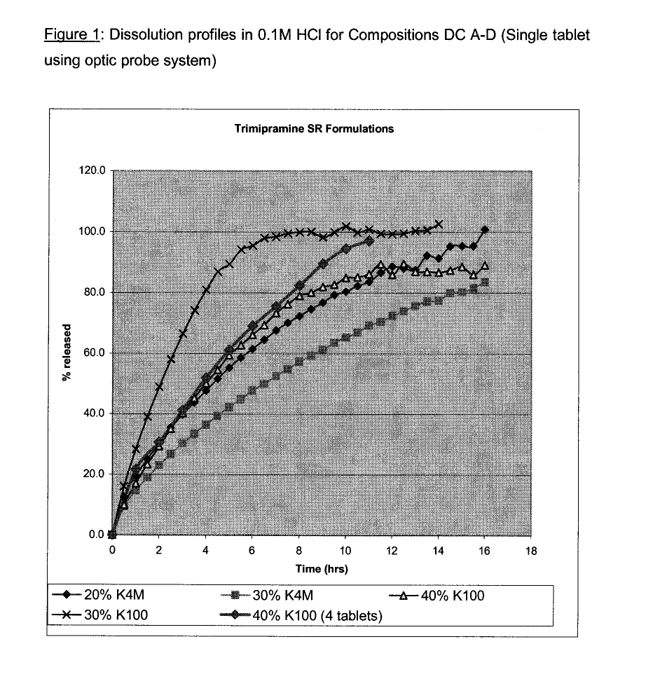

Tables 5, 6, 7, 8 below shows the Trimipramine release percentages up to 16 hours from the DC tablet of compositions A-D respectively in 0.1M HCl. The corresponding graph of Trimipramine release over time using a single tablet and the optic probe system are shown in FIG. 1.

TABLE 5
Trimipramine 100 mg optic probe DJ1/57/20% K4M
(DC A) release percentages (R %) over time

| Time | 0 | 0.5 | 1 | 1.5 | 2 | 2.5 | 3 | 3.5 | 4 | 4.5 | 5 |
|---|---|---|---|---|---|---|---|---|---|---|---|
| R % | 0.0 | 12.6 | 19.4 | 25.1 | 30.2 | 35.4 | 39.7 | 43.7 | 47.7 | 51.6 | 55.2 |
| Time | 5.5 | 6 | 6.5 | 7 | 7.5 | 8 | 8.5 | 9 | 9.5 | 10 | 10.5 |
| R % | 58.6 | 61.5 | 64.4 | 67.5 | 70.1 | 72.4 | 74.6 | 76.8 | 79.3 | 80.3 | 82.3 |
| Time | 11 | 11.5 | 12 | 12.5 | 13 | 13.5 | 14 | 14.5 | 15 | 15.5 | 16 |
| R % | 83.7 | 86.6 | 88.7 | 88.0 | 87.6 | 92.3 | 91.4 | 95.3 | 95.4 | 95.5 | 100.8 |

TABLE 6
Trimipramine 100 mg optic probe DJ1/58/30% K4M
(DC B) release percentages (R %) over time

| Time | 0 | 0.5 | 1 | 1.5 | 2 | 2.5 | 3 | 3.5 | 4 | 4.5 | 5 |
|---|---|---|---|---|---|---|---|---|---|---|---|
| R % | 0.0 | 9.4 | 14.6 | 19.0 | 23.0 | 26.7 | 30.2 | 33.3 | 36.4 | 39.3 | 42.2 |
| Time | 5.5 | 6 | 6.5 | 7 | 7.5 | 8 | 8.5 | 9 | 9.5 | 10 | 10.5 |
| R % | 44.9 | 47.7 | 49.9 | 52.6 | 54.8 | 57.3 | 59.3 | 61.3 | 63.5 | 65.3 | 66.9 |
| Time | 11 | 11.5 | 12 | 12.5 | 13 | 13.5 | 14 | 14.5 | 15 | 15.5 | 16 |
| R % | 69.2 | 70.5 | 72.4 | 74.0 | 75.8 | 77.2 | 77.4 | 80.0 | 80.3 | 81.6 | 83.6 |

TABLE 7
Trimipramine 100 mg optic probe DJ1/59/40% K100
(DC C) release percentages (R %) over time

| Time | 0 | 0.5 | 1 | 1.5 | 2 | 2.5 | 3 | 3.5 | 4 | 4.5 | 5 |
|---|---|---|---|---|---|---|---|---|---|---|---|
| R % | 0.0 | 10.0 | 17.0 | 23.3 | 29.3 | 35.0 | 40.2 | 45.3 | 50.0 | 54.5 | 59.3 |
| Time | 5.5 | 6 | 6.5 | 7 | 7.5 | 8 | 8.5 | 9 | 9.5 | 10 | 10.5 |
| R % | 62.7 | 66.1 | 69.3 | 73.3 | 76.2 | 79.0 | 80.1 | 81.9 | 82.7 | 84.9 | 85.2 |
| Time | 11 | 11.5 | 12 | 12.5 | 13 | 13.5 | 14 | 14.5 | 15 | 15.5 | 16 |
| R % | 86.2 | 89.4 | 86.0 | 89.6 | 87.0 | 87.0 | 86.7 | 87.4 | 88.6 | 86.1 | 89.1 |

TABLE 8
Trimipramine 100 mg optic probe DJ1/62/30% K100
(DC D) release percentages (R %) over time

| Time | 0 | 0.5 | 1 | 1.5 | 2 | 2.5 | 3 | 3.5 | 4 | 4.5 |
|---|---|---|---|---|---|---|---|---|---|---|
| R % | 0.0 | 16.0 | 28.3 | 39.0 | 48.9 | 58.0 | 66.4 | 74.1 | 81.0 | 87.0 |
| Time | 5 | 5.5 | 6 | 6.5 | 7 | 7.5 | 8 | 8.5 | 9 | 9.5 |
| R % | 89.5 | 94.2 | 95.6 | 97.9 | 98.4 | 99.5 | 100.0 | 100.0 | 98.2 | 99.9 |
| Time | 10 | 10.5 | 11 | 11.5 | 12 | 12.5 | 13 | 13.5 | 14 | |
| R % | 101.8 | 99.8 | 100.8 | 99.4 | 99.3 | 99.5 | 100.4 | 100.7 | 102.6 | |

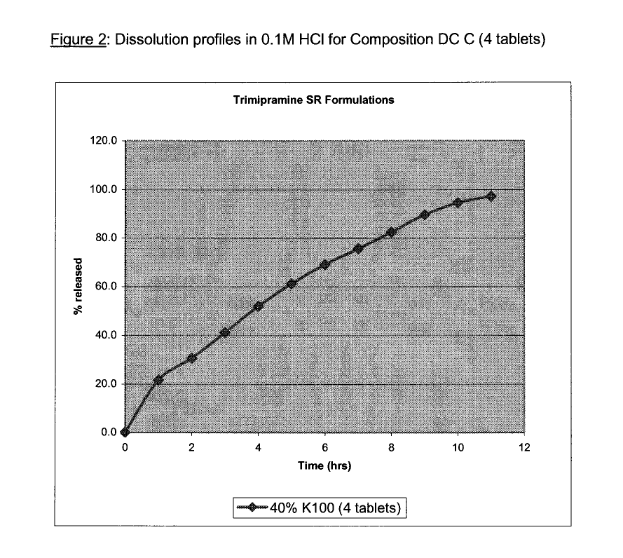

Table 9 shows the dissolution results of 4 tablets of Composition C (40% K100). FIG. 2 shows a graph of the mean (of 4 tablets) of Trimipramine release over time.

TABLE 9
Trimipramine 100 mg DJ1/59/40% K100 (DC C) release
percentages at different times using 4 tablets

| | Time (hours) | | | | | | | | | | | | |
|---|---|---|---|---|---|---|---|---|---|---|---|---|---|
| | 0 | 1 | 2 | 3 | 4 | 5 | 6 | 7 | 8 | 9 | 10 | 11 | 24 |
| Tablet 1 | 0.0 | 24.4 | 31.9 | 43.0 | 54.7 | 64.0 | 72.6 | 78.7 | 85.7 | 93.0 | 97.7 | 99.4 | 104.2 |
| Tablet 2 | 0.0 | 18.7 | 29.4 | 39.4 | 49.1 | 58.3 | 65.5 | 72.4 | 79.2 | 86.1 | 91.4 | 94.9 | 101.1 |
| Tablet 3 | 0.0 | 19.9 | 30.4 | 40.7 | 50.3 | 59.2 | 66.9 | 74.7 | 81.7 | 88.8 | 95.3 | 99.6 | 104.1 |
| Tablet 4 | 0.0 | 24.3 | 31.4 | 42.1 | 52.2 | 61.5 | 70.0 | 78.0 | 84.8 | 92.6 | 98.2 | 101.9 | 105.5 |
| Average | 0.0 | 21.6 | 30.7 | 41.2 | 51.9 | 61.1 | 69.1 | 75.6 | 82.5 | 89.6 | 94.5 | 97.1 | 102.7 |

The scope of the invention is defined by the following claims.

1.-30. (canceled)

31. An orally deliverable composition for the once daily (OD) administration of trimipramine, the composition comprising a therapeutically effective amount of trimipramine dispersed in a water-swellable polymeric matrix, wherein the matrix comprises at least one pharmaceutically acceptable excipient comprising a water-swellable polymer, wherein the composition exhibits an in vitro release profile wherein from about 10 to about 50% of the trimipramine is dissolved within 3 hours after placement in a standard dissolution test.

32. The composition of claim 31, which exhibits an in vitro release profile wherein from about 15 to about 45% of the trimipramine is dissolved within 3 hours after placement in a standard dissolution test.

33. The composition of claim 31, which exhibits an in vitro release profile wherein from about 25 to about 100% of the trimipramine is dissolved within 8 hours after placement in a standard dissolution test.

34. The composition of claim 33, wherein from about 50 to about 100% of the trimipramine is dissolved within 8 hours after placement in a standard dissolution test.

35. The composition of claim 31, that exhibits an in vitro dissolution rate after placement in a standard dissolution test wherein:
from about 5 to about 40% of the trimipramine is released after 2 hours;
from about 15 to about 70% of the trimipramine is released after 4 hours; and
50% or more of the trimipramine is released after 8 hours.

36. The composition of claim 31, that exhibits an in vivo trimipramine plasma absorption profile following single dose oral administration wherein the time for about 50% of the trimipramine to be absorbed into the plasma is from about 2 to about 12 hours.

37. The composition of claim 31, that exhibits an in vivo release profile wherein:
from about 5 to about 40% of the trimipramine is released within 2 hours following administration;
from about 15 to about 70% of the trimipramine is released within 4 hours following administration; and
50% or more of the trimipramine is released within 8 hours following administration.

38. The composition of claim 31, that exhibits a trimipramine Cₘₐₓ value following oral administration that is from about 20 to about 80% of the Cₘₐₓ value achieved using a conventional immediate release (IR) dosage form of trimipramine when administered orally at an identical dose.

39. The composition of claim 31, wherein the composition provides a ratio of the peak plasma concentration (Cₘₐₓ) of trimipramine to the plasma concentration of trimipramine 24 hours after administration (C₂₄) following oral administration of less than about 4:1.

40. The composition of claim 31, wherein the water-swellable polymer comprises a cellulose polymer or derivative thereof.

41. The composition of claim 40, wherein the cellulose polymer or derivative thereof comprises an alkyl-substituted cellulosic polymer.

42. The composition of claim 41, wherein the alkyl-substituted cellulosic polymer is selected from methylcellulose, hydroxymethyl-cellulose, hydroxyethyl cellulose, hydroxypropylcellulose, hydroxypropylmethylcellulose, and carboxymethylcellulose.

43. The composition of claim 42, wherein the alkyl-substituted cellulosic polymer is hydroxypropylmethylcellulose.

44. The composition of claim 31, wherein the matrix further comprises a filler.

45. The composition of claim 44, wherein the filler comprises a water-insoluble filler.

46. The composition of claim 45, wherein the water-insoluble filler is selected from silicon dioxide, titanium dioxide, talc, alumina, starch, kaolin, polacrilin potassium, powdered cellulose, microcrystalline cellulose, and combinations thereof.

47. The composition of claim 46, wherein the water-insoluble filler comprises microcrystalline cellulose.

48. The composition of claim 31, wherein the matrix further comprises a lubricant.

49. The composition of claim 48, wherein the lubricant is selected from calcium stearate, glycerol behenate, magnesium stearate, mineral oil, polyethylene glycol, sodium stearylfumarate, stearic acid, talc, vegetable oil, zinc stearate, and combinations thereof.

50. The composition of claim 49, wherein the lubricant comprises sodium stearylfumarate.

51. The composition of claim 31, wherein the trimipramine is in the form of a pharmaceutically acceptable organic salt of trimipramine.

52. The composition of claim 51, wherein the organic salt is prepared from organic acids selected from acetic, trifluoroacetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethanedisulfonic, oxalic, isethionic and HO₂C—(CH₂)ₙ—CO₂H (where n=0-4); and salts prepared from amino acids selected from arginate, asparginate and glutamate.

53. The composition of claim 52, wherein the organic salt is trimipramine maleate.

54. The composition of claim 31, further comprising at least one pharmaceutically active agent in addition to trimipramine.

55. The composition of claim 54, in which the at least one additional pharmaceutically active agent is selected from atypical antipsychotic agents (e.g. olanzapine, quetiapine, risperidone, amisulpride, clozepine, chlorpromazine, or haloperidol decanoate), antiparkinsonian agents (e.g. L-DOPA, Dopamine Agonists, anticholinergic drugs), sedatives (e.g. a benzodiazepine sedative or non-barbituate sedative), anxiolytics (e.g. benzodiazepines such as lorazepam, chlordiazepoxide, oxazepam, clorazepate, diazepam, and alprazolam), antidepressants (e.g. tricyclic antidepressants (such as amitriptyline, imipramine, doxepin, and clomipramine), monoamine oxidase A or B inhibitors (such as phenelzine and tranylcypromine), tetracyclic antidepressants (e.g. maprotiline), serotonin re-uptake inhibitors (such as fluoxetine, cipramil, S-cipramil, paroxetine, and sertraline hydrochloride, serotonin) and nor adrenaline reuptake inhibitors (such as venlafaxine and duloxetine), or adrenaline reuptake inhibitors (such as reboxetine and viloxazine), and mood stabilisers (e.g. lamotrigine, lithium, valproate, carbamazepine, oxcarbazepine).

56. A method of treating a subject with at least one of a neurological condition and a psychiatric condition, the method comprising administering to the subject a composition as defined in claim 31.

57. A method of treatment at least one of a neurological condition and a psychiatric condition, the method comprising administration of a composition as defined in claim 31 to a subject in need of such treatment.

58. A method of prophylactically heating a subject susceptible to at least one of a neurological condition and psychiatric condition, the method comprising administration of a composition as defined in claim 31, to the susceptible subject.

59. The method of claim 56 wherein the treatment is associated with the effect of trimipramine on neurotransmitter pathways in the brain.

60. The method of claim 56, wherein the at least one of the neurological condition and the psychiatric condition is associated with dopamine receptors.

61. The method of claim 56, wherein the at least one of the neurological condition and the psychiatric condition is selected from all psychoses and neuroses, including all Depressive Disorders and Symptoms, Primary and Secondary Insomnia, Schizophrenia and Bipolar Disorders, Schizoaffective disorders, Anxiety disorders, obsessive compulsive disorder, Post Traumatic Stress Disorder, Personality Disorder and Borderline Personality Disorder, all types of dementia and/or cognitive impairment (e.g. mild cognitive impairment of the elderly); psychiatric complications of stroke (including haemorrhagic and ischaemic and sequelae), epilepsy, transient ischaemic attacks, traumatic brain injury, Parkinsons disease, Huntingtons disease, amytrophic lateral sclerosis; neuropathic pain, idiopathic pain, all psychoses (such as degenerative Depression and catatonia), all addictions, (e.g. addiction to alcohol, nicotine and opiates), all eating disorders including bulimia and anorexia, affective disorders including ADHD (attention deficit hyperactivity disorder), personality disorders (including borderline personality disorders), sleep disorders (including jet lag and insomnia), Downs syndrome, meningitis, central nervous system vasculitis, leukodystrophies and adrenoleukodystrophies (including Alexander's disease, Canavan's disease, cerebrotendinous xanthomatosis, Krabbes and metachromatic LD), fatigue, hypoglycaemia, encephalopathy, (such as hepatic and septic encephalopathy), tumours of the brain and spinal cord (including primary tumours of glial, neuronal, schwann cell, pinealcyte, meningioma, melanoma, sarcoma, lymphoma and multiple systemic systemic malignancies which metasize), cerebellar degeneration and ataxias (e.g. Friedrich's ataxia, cerebellar cortical atazia, complicated cerebellar ataxia, which includes olivopontocerebellar degeneration, spinocerebellar disease, dentatorubral degeneration and autosomal dominant ataxias) vertigo, vestibular system damage, cochlear disorders such as tinnitus, nystagmus, peripheral neuropathy, (e.g. polyneuropathy, polyradiculopathy, motor neuronopathy, sensor neuronopathy, multiple mononeuropathy and plexopathies), metabolic bone diseases, osteoporosis, pulmonary disorders, (such as pulmonary edema, neurogenic pulmonary edema, bronchial asthma, adult respiratory distress syndrome (ARDS) and pulmonary cell death by apoptosis or necrosis), obesity and complications thereof, diabetes and prediabetes, and combinations thereof.

62. The method of claim 56, wherein the at least one of the neurological condition and the psychiatric condition comprises depressive disorder and symptoms thereof, primary and secondary insomnia.

63. The method of claim 56, wherein the at least one of the neurological condition and the psychiatric condition comprises an anxiety disorder.

64. A medicament for oral administration to provide the controlled release of trimipramine to a subject in need thereof, wherein the medicament comprises the composition of claim 31.
